(19) United States
(12) Patent Application Publication
(10) Pub. No.: US 2023/0406847 A1
(43) Pub. Date: Dec. 21, 2023

(54) LIGANDS OF PROSTATE SPECIFIC MEMBRANE ANTIGEN (PSMA) CONTAINING HETEROAROMATIC LINKER BUILDING BLOCKS

(71) Applicant: ABX ADVANCED BIOCHEMICAL COMPOUNDS GMBH, Radeberg (DE)

(72) Inventors: Alexander HOEPPING, Dresden (DE); Hans-Joachim LANKAU, Weinböhla (DE); Ronny HESSE, Dresden (DE); Klaus KOPKA, Dresden (DE); Ulrike BAUDER WÜST, Schriesheim (DE); Christian LIS, Dresden (DE); René SMITS, Dresden (DE); Jan MOLLITOR, Radeberg (DE); Kristine SCHEIBE, Dresden (DE); Alexandra GEISSLER, München (DE)

(73) Assignee: ABX ADVANCED BIOCHEMICAL COMPOUNDS GMBH, Radeberg (DE)

(21) Appl. No.: 18/036,584

(22) PCT Filed: Nov. 11, 2021
(86) PCT No.: PCT/EP2021/081404

§ 371 Date: May 11, 2023

(30) Foreign Application Priority Data
Nov. 12, 2020 (EP) 20207224.5

(51) Int. Cl. C07D 413/14; C07D 413/12; C07D 401/12; C07D 409/12; C07D 417/12; A61K 51/04

(52) U.S. Cl. CPC C07D 413/14; C07D 413/12; C07D 401/12; C07D 409/12; C07D 417/12; A61K 51/0497; A61K 2121/00; A61K 2123/00

(57) ABSTRACT

The present invention relates to novel compounds that bind to the prostate-specific membrane antigen (PSMA)-binding and their use in the diagnosis and treatment of certain diseases where PSMA is upregulated.

### TECHNICAL FIELD

The present invention generally relates to novel compounds that bind to the prostate-specific membrane antigen (PSMA)-binding and their use in the diagnosis and treatment of certain diseases where PSMA is upregulated.

### BACKGROUND OF THE INVENTION

Once metastasized, prostate cancer (PC) becomes one of the most aggressive types of tumors. It affects at least 2 million men in the US and 4 million men in Europe.

Estimated new cases and deaths from prostate cancer in the United States in 2014: New cases: 233,000; Deaths: 29,480 (http://www.cancer.gov/cancertopics/types/prostate—available in August 2020).

Estimated new cases and deaths from prostate cancer in Europe in 2012: New cases: 400,000; Deaths: 92,000 (http://eco.iarc.fr/eucan/CancerOne.aspx?Cancer=29&Gender=1—available in August 2020).

After initial response to androgen deprivation therapy by pharmaceutical or surgical castration in almost all men most patients with metastatic disease sooner or later develop castration resistant prostate cancer (mCRPC). During the past decade new therapeutic options, such as new antihormonal therapies (abiraterone acetate and enzalutamide), systemically administered radiopharmaceuticals (RPs), including ²²³RaCl₂, immunotherapy (sipuleucel-T) and the 2^{nd} line chemotherapy cabazitaxel were approved for mCRPC patients. Despite these developments, mCRPC will claim the lives of more than 250,000 men worldwide each year. Globally, >900,000 new cases of PC were diagnosed in 2010. According to the World Cancer Research Fund International, it is predicted that the number of new PC cases will almost double (1.7 million) by 2030. Alternative therapeutic options such as systemic radiation therapy for men with mCRPC are thus urgently needed.

Targeted systemic radiation therapy offers the possibility to treat the cancer lesions in a specific and tumor-selective manner by addressing cell surface receptors mainly expressed on malignant cells.

The prostate-specific membrane antigen (PSMA) is an ideal target for the therapy of prostate cancer because it is highly expressed on the surface of prostate epithelial cells and it is strongly upregulated (100-1000 fold) in PC with metastatic and hormone-ref ractory cells meeting the clinical requirements for an effective therapy of metastatic PC. Because of the low expression levels in healthy tissues, PSMA has the potential for high-dose endoradiation therapy with minimized radioactivity-related side effects. A highly effective treatment can be realised using radiolabeld small-molecule ligands of PSMA. After binding PSMA ligands are internalized via clathrin-coated pits resulting in an effective uptake of the bound radioligand molecule into the prostate epithelial cell.

The PSMA-binding ligands are described, for example, in the following publications:
Pomper M G, Zhang J, Kozikowski A P, Musachio J L. Imaging agents and methods of imaging NAALADase of PSMA. U.S. Patent Application Publication No. 2004/0054190.
Pomper, M. Pomper, Martin Gilbert; Ray, Sangeeta; Mease, Ronnie C.; Foss, Catherine. Labeled inhibitors of prostate specific membrane antigen (PSMA), biological evaluation, and use as imaging agents. PCT Patent Application Publication No. WO 2009002529.
Pomper M G, Mease R C, Chen Y. Preparation of glutamic acid heterodimer prostate-specific membrane antigen PSMA binding agents for therapeutic and imaging use. PCT Patent Application Publication No. WO 2010014933.
Pomper, Martin G.; Mease, Ronnie C.; Ray, Sangeeta; Chen, Ying. PSMA- targeting compounds and uses thereof. PCT Patent Application Publication No. WO 2010108125.
Pomper, Martin G. Mease, Ronnie C., Ray, Sangeeta, Shallal, Hassan. Homomultivalent and heteromultivalent inhibitors of prostate specific membrane antigen (PSMA) and uses thereof. PCT Patent Application Publication No. WO 2013082338.
Babich J W, Zimmerman C N, Maresca K P. Heterodimers of glutamic acid. U.S. Patent Application Publication No. 2008/0193381.
Babich J W, Zimmerman C, Joyal J L, Lu G. Radiolabeled prostate specific membrane antigen inhibitors U.S. Patent Application Publication No. 2013/0034494.
Low P S, Chelvam V, Kim Y. PSMA binding ligand-linker conjugates and methods for using. PCT Patent Application Publication Nos. WO 2011/106639, WO 2010/045598, and WO 2009/026177.
Pomper M G, Mease R C, Chen Y, Ray S, Zalutsky M, Vaidyanathan G. Preparation of PSMA targeted radiohalogenated urea peptidomimetics for cancer therapy. PCT Patent Application Publication No. WO 2017070482.
Ray S, Pomper M G. Prostate-specific membrane antigen targeted high affinity agents for endoradiotherapy of prostate cancer. PCT Patent Application Publication No. WO 2017165473. Kiess A P, Minn I, Vaidyanathan G, et al. (2S)-2-(3-(I-Carboxy-5-(4-2IIAt-Astatobenzamido)Pentyl)Ureido)-Pentanedioic Acid for PSMA-Targeted alpha-Particle Radiopharmaceutical Therapy. J Nucl Med. 2016; 57: 1569-1575.
Mease R, Kang C, Kumar V, et al. Small Molecule Radiohalogenated (1251/21 IAt), DOTA Containing PSMA Inhibitors: Metal Complexation and Competing Inhibitor Improve Biodistribution in Mice. Journal of Nuclear Medicine. 20I8; 59:537

The present invention describes PSMA-binding molecules that show significantly higher internalisation rates than those known from the prior art.

### SUMMARY OF THE INVENTION

In one aspect, the present invention relates to PSMA-binding molecules of Formula I

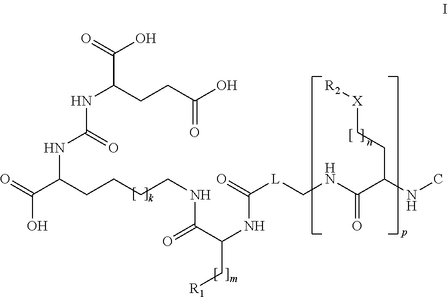

wherein
C is represented by any of the structures selected from the group consisting of
1,4,7,10-tetraazacyclododecane-1,4,7,10-tetraacetic acid (DOTA),
N,N″-bis[2-hydroxy-5-(carboxyethyl)benzyl]ethylenediamine-N,N″-diacetic acid (HBED-CC),
1,4-bis(carboxymethyl)-6-[bis(carboxymethyl)]amino-6-methylperhydro-1,4-diazepine (AAZTA),
1,4,7-triazacyclononane-1,4,7-triacetic acid (NOTA),
2-(4,7,10-tris(carboxymethyl)-1,4,7,10-tetraazacyclododecan-1-yl)pentanedioic acid (DOTAGA),
N⁴-[5-[[4-[[5-(acetylhydroxyamino)pentyl]amino]-1,4-dioxobutyl]hydroxyamino]pentyl]-N¹ -(5-aminopentyl)-N¹-hydroxybutanediamide (DFO),
N¹-[5-(acetylhydroxyamino)pentyl]-N²⁶-(5-aminopentyl)-N²⁶,5,16-trihydroxy-4,12,15,23-tetraoxo-5,11,16,22-tetraazahexacosanediamide (DFO*) or
5-[(2-mercapto-2-methylpropyl)[2-[(2-mercapto-2-methylpropyl)amino]ethyl]amino]pentanoic acid (BAT),
   L is C1-C5-heteroaryl, optionally substituted with C1-C6 alkyl,
   X is NH or CH₂,
   k is 0, 1 or 2,
   m is an integer from 1 to 7,
   n is an integer from 0 to 6,
   p is 0 or 1,
   R₁ is phenyl, phenyl-NH—, benzothiophenyl or naphthyl,
   wherein phenyl and phenyl-NH— is optionally substituted with 1, 2 or 3 groups each independently selected from halo, OH and NO₂, and
   R₂ is aryl, aryl-C(O)—, or heteroaryl-C(O)—, optionally substituted with 1, 2 or 3 groups each independently selected from halo, OH, C1-C6-alkyl, OCH₃ and NO₂,
   wherein C1-C5-heteroaryl contains 1, 2, 3 or 4 heteroatoms, each independently selected from N, O or S,
   or a pharmaceutically acceptable salt thereof or a solvate thereof or a solvate of a salt thereof.

It has been surprisingly found that the compounds of the present invention, which contain heteroaromatic linker building blocks, exhibit significantly higher internalisation rates than those known from the prior art.

In one embodiment L is C1-C5-heteroaryl, containing 1, 2, 3 or 4 heteroatoms each independently selected from N, O or S, optionally substituted by C1-C6-alkyl. In a preferred embodiment L is C3-C5-heteroaryl, containing 1, 2 or 3 heteroatoms each independently selected from N, O or S, optionally substituted by C1-C6-alkyl.

In one embodiment L is selected from the group consisting of isoxazolyl, pyridyl, thiazolyl and thiophenyl, optionally substituted with C1-C6 alkyl. In one embodiment L is selected from the group consisting of isoxazolyl, thiazolyl and thiophenyl, optionally substituted with C1-C6 alkyl. In one embodiment L is isoxazolyl, optionally substituted with C1-C6 alkyl. In one embodiment L is pyridyl, optionally substituted with C1-C6 alkyl. In one embodiment L is thiazolyl, optionally substituted with C1-C6 alkyl. In one embodiment L thiophenyl, optionally substituted with C1-C6 alkyl.

In one embodiment C is represented by any of the structures selected from the group consisting of
1,4,7,10-tetraazacyclododecane-1,4,7,10-tetraacetic acid (DOTA),
N⁴-[5-[[4-[[5-(acetylhydroxyamino)pentyl]amino]-1,4-dioxobutyl]hydroxyamino]pentyl]-N¹-(5-aminopentyl)-N¹-hydroxy-succinamide (DFO),
N-[5-({3-[5-(Acetyl-hydroxy-amino)-pentylcarbamoyl]-propionyl}-hydroxy-amino)-pentypl]-N′[5-(10-amino-5-hydroxy-4-oxo-decanoylamino)-pentyl]-N′-hydroxy- succinamide (DFO*), or
5-[(2-mercapto-2-methylpropyl)[2-[(2-mercapto-2-methylpropyl)amino]ethyl]amino]-pentanoic acid (BAT).

In one embodiment C is 1,4,7,10-tetraazacyclododecane-1,4,7,10-tetraacetic acid (=DOTA).

The chelator C may be connected to the NH group via a C(O) group, in particular via a C(O) group of a carboxylic acid end.

In one embodiment R₁ is phenyl, phenyl-NH—, benzothiophenyl or naphthyl, wherein phenyl and phenyl-NH— is optionally substituted with 1, 2 or 3 groups each independently selected from halo, OH and NO₂. In one embodiment R₁ is benzothiophenyl or naphthyl.

In one embodiment R₂ is aryl, aryl-C(O)—, or heteroaryl-C(O)—, optionally substituted with 1, 2 or 3 groups each independently selected from halo, OH, C1-C6-alkyl, OCH₃ and NO₂.

In one embodiment m is 1.

In one embodiment the subject-matter of the invention is a compound of Formula I that is a compound of Formula II

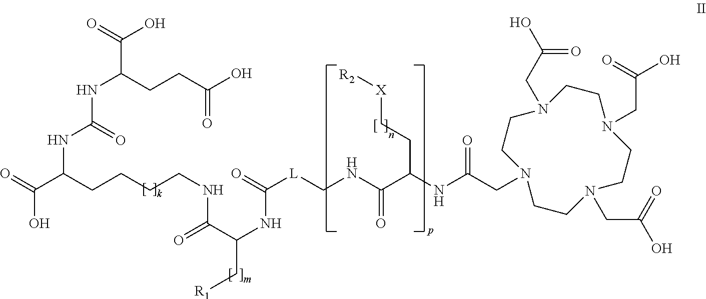

wherein
L is C1-C5-heteroaryl, optionally substituted with C1-C6 alkyl,
X is NH or CH₂,
k is 0, 1 or 2,
m is an integer from 1 to 7,
n is an integer from 0 to 6,
p is 0 or 1,
R₁ is phenyl, phenyl-NH—, benzothiophenyl or naphthyl,
wherein phenyl and phenyl-NH— is optionally substituted with 1, 2 or 3 groups each independently selected from halo, OH and NO₂, and
R₂ is aryl, aryl-C(O)—, or heteroaryl-C(O)—, optionally substituted with 1, 2 or 3 groups each independently selected from halo, OH, C1-C5-alkyl, OCH₃ and NO₂,
wherein C1-C5-heteroaryl contains 1, 2, 3 or 4 heteroatoms, each independently selected from N, O or S,
or a pharmaceutically acceptable salt thereof or a solvate thereof or a solvate of a salt thereof.

In one embodiment the subject-matter of the invention is a compound of Formula I,
wherein C is represented by any of the structures selected from the group consisting of
1,4,7,10-tetraazacyclododecane-1,4,7,10-tetraacetic acid (DOTA),
N,N″-bis[2-hydroxy-5-(carboxyethyl)benzyl]ethylenediamine-N,N″-diacetic acid (HBED-CC),
1,4-bis(carboxymethyl)-6-[bis(carboxymethyl)]amino-6-methylperhydro-1,4-diazepine (AAZTA),
1,4,7-triazacyclononane-1,4,7-triacetic acid (NOTA),
2-(4,7,10-tris(carboxymethyl)-1 ,4,7,10-tetraazacyclododecan-1 -yl)pentanedioic acid (DOTAGA),
N⁴-[5-[[4-[[5-(acetylhydroxyamino)pentyl]amino]-1,4-dioxobutyl]hydroxyamino]pentyl]-N¹-(5-aminopentyl)-N¹-hydroxybutanediamide (DFO),
N¹-[5-(acetylhydroxyamino)pentyl]-N²⁶ -(5-aminopentyl)-N^{26,5,16}-trihydroxy-4,12,15,23-tetraoxo-5,11,16,22-tetraazahexacosanediamide (DFO*) or
5-[(2-mercapto-2-methylpropyl)[2-[(2-mercapto-2-methylpropyl)amino]ethyl]amino]pentanoic acid (BAT),
   L is selected from the group consisting of isoxazolyl, pyridyl, thiazolyl and thiophenyl, optionally substituted with C1-C6 alkyl, in particular from the group consisting of isoxazolyl, thiazolyl and thiophenyl, optionally substituted with C1-C6 alkyl,
   X is NH or CH₂,
   k is 0, 1 or 2,
   m is an integer from 1 to 7,
   n is an integer from 0 to 6,
   p is 0 or 1,
   R₁ is phenyl, phenyl-NH—, benzothiophenyl or naphthyl,
   wherein phenyl and phenyl-NH— is optionally substituted with 1, 2 or 3 groups each independently selected from halo, OH and NO₂, and
   R₂ is aryl, aryl-C(O)—, or heteroaryl-C(O)—, optionally substituted with 1, 2 or 3 groups each independently selected from halo, OH, C1-C6-alkyl, OCH₃ and NO₂,
   or a pharmaceutically acceptable salt thereof or a solvate thereof or a solvate of a salt thereof.

In one embodiment the subject-matter of the invention is a compound of Formula I that is a compound of Formula III

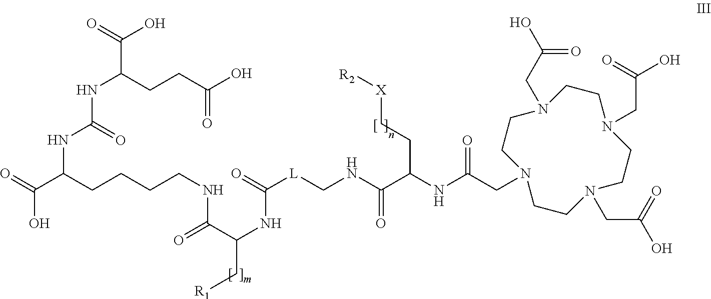

wherein
X is NH or CH₂,
m is an integer from 1 to 7,
n is an integer from 0 to 6,
R₁ is phenyl, phenyl-NH—, benzothiophenyl or naphthyl,
wherein phenyl and phenyl-NH— is optionally substituted with 1, 2 or 3 groups each independently selected from halo, OH and NO₂,
R₂ is aryl, aryl-C(O)—, or heteroaryl-C(O)—, optionally substituted with 1, 2 or 3 groups each independently selected from halo, OH, C1-C6-alkyl, OCH₃ and NO₂, and
L is selected from the group consisting of isoxazolyl, pyridyl, thiazolyl and thiophenyl, optionally substituted with C1-C6 alkyl,
or a pharmaceutically acceptable salt thereof or a solvate thereof or a solvate of a salt thereof.

In one embodiment the subject-matter of the invention is a compound of Formula I that is a compound of Formula III

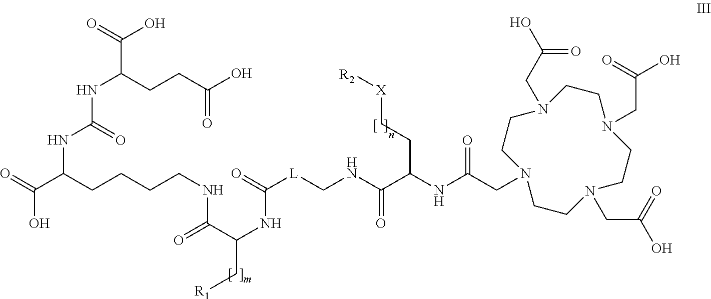

wherein
X is NH or CH₂,
m is an integer from 1 to 7,
n is an integer from 0 to 6,
R₁ is phenyl, phenyl-NH—, benzothiophenyl or naphthyl,
wherein phenyl and phenyl-NH— is optionally substituted with 1, 2 or 3 groups each independently selected from halo, OH and NO₂,
R₂ is aryl, aryl-C(O)—, or heteroaryl-C(O)—, optionally substituted with 1, 2 or 3 groups each independently selected from halo, OH, C1-C6-alkyl, OCH₃ and NO₂, and
L is selected from the group consisting of isoxazolyl, thiazolyl and thiophenyl, optionally substituted with C1-C6 alkyl,
or a pharmaceutically acceptable salt thereof or a solvate thereof or a solvate of a salt thereof.

In one embodiment the subject-matter of the invention is a compound of Formula I that is a compound of Formula IV

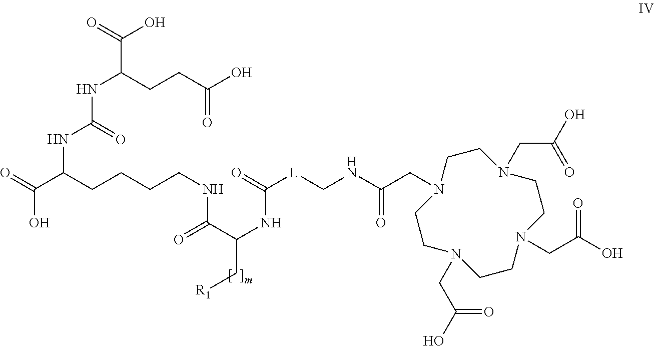

wherein
m is an integer from 1 to 7,
R₁ is phenyl, phenyl-NH—, benzothiophenyl or naphthyl,
wherein phenyl and phenyl-NH— is optionally substituted with 1, 2 or 3 groups each independently selected from halo, OH and NO₂, and
L is selected from the group consisting of isoxazolyl, pyridyl, thiazolyl and thiophenyl, optionally substituted with C1-C6 alkyl,
or a pharmaceutically acceptable salt thereof or a solvate thereof or a solvate of a salt thereof.

In one embodiment the subject-matter of the invention is a compound of Formula I that is a compound of Formula IV

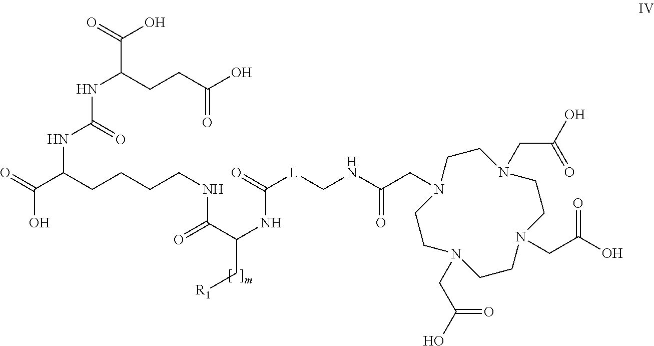

wherein
m is an integer from 1 to 7,
R₁ is phenyl, phenyl-NH—, benzothiophenyl or naphthyl,
wherein phenyl and phenyl-NH— is optionally substituted with 1, 2 or 3 groups each
independently selected from halo, OH and NO₂, and
L is selected from the group consisting of isoxazolyl, thiazolyl and thiophenyl, optionally substituted with C1-C6 alkyl,
or a pharmaceutically acceptable salt thereof or a solvate thereof or a solvate of a salt thereof.

In one embodiment the subject-matter of the invention is a compound of Formula I that is a compound of Formula V

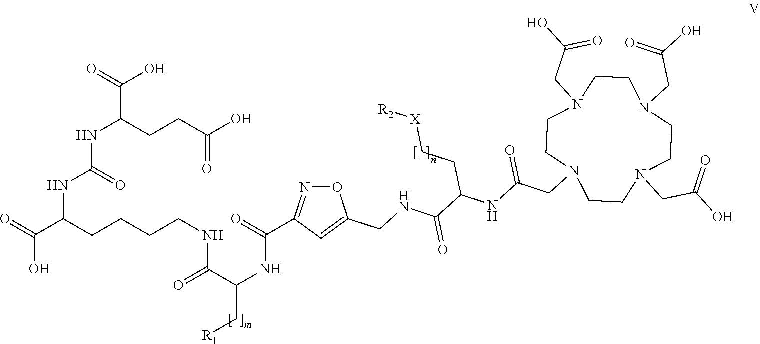

wherein
X is NH or CH₂,
m is an integer from 1 to 7,
n is an integer from 0 to 6,
R₁ is phenyl, phenyl-NH—, benzothiophenyl or naphthyl,
wherein phenyl and phenyl-NH— is optionally substituted with 1, 2 or 3 groups each independently selected from halo, OH and NO₂, and
R₂ is aryl, aryl-C(O)—, or heteroaryl-C(O)—, optionally substituted with 1, 2 or 3 groups each independently selected from halo, OH, C1-C6-alkyl, OCH₃ and NO₂,
or a pharmaceutically acceptable salt thereof or a solvate thereof or a solvate of a salt thereof.

In one embodiment the subject-matter of the invention is a compound of Formula I that is a compound of Formula VI

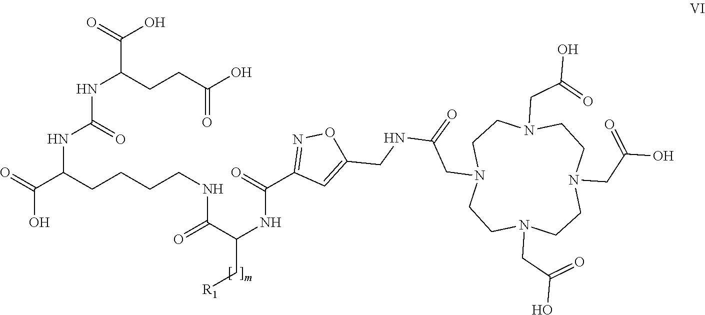

wherein
m is an integer from 1 to 7, and
R₁ is phenyl, phenyl-NH—, benzothiophenyl or naphthyl,
wherein phenyl and phenyl-NH— is optionally substituted with 1, 2 or 3 groups each independently selected from halo, OH and NO₂,
or a pharmaceutically acceptable salt thereof or a solvate thereof or a solvate of a salt thereof.

In one embodiment the subject-matter of the invention is a compound according to any of the preceding embodiments, wherein R₁ is benzothiophenyl or naphthyl and m is 1, or a pharmaceutically acceptable salt thereof or a solvate thereof or a solvate of a salt thereof.

In one embodiment, the subject-matter of the present invention further relates to a compound of Formula I according to any of the preceding embodiments, with the proviso that when p is 0 and L is isoxazolyl, R₁ is not naphthyl.

In one embodiment the subject-matter of the invention is a compound selected from the list of the following compounds:

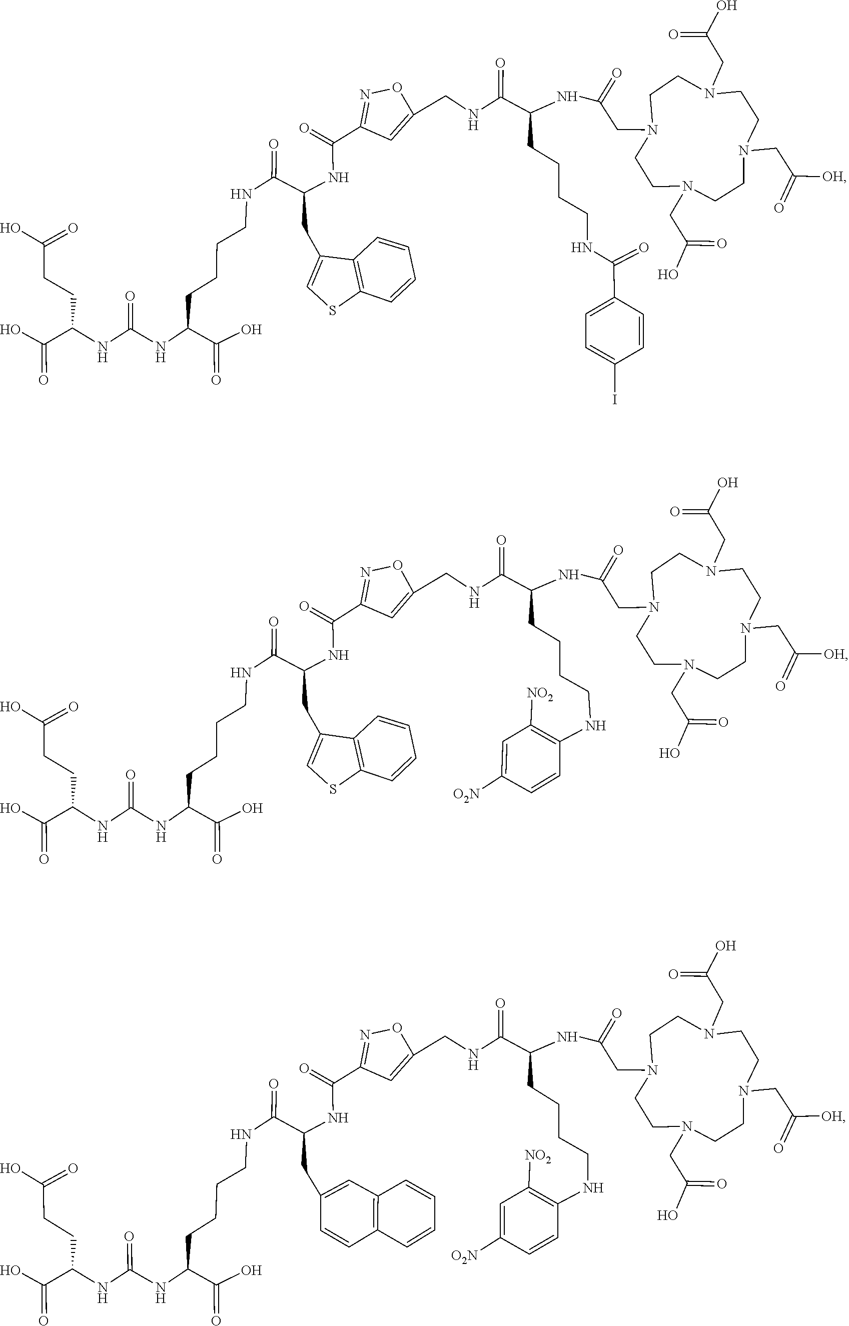

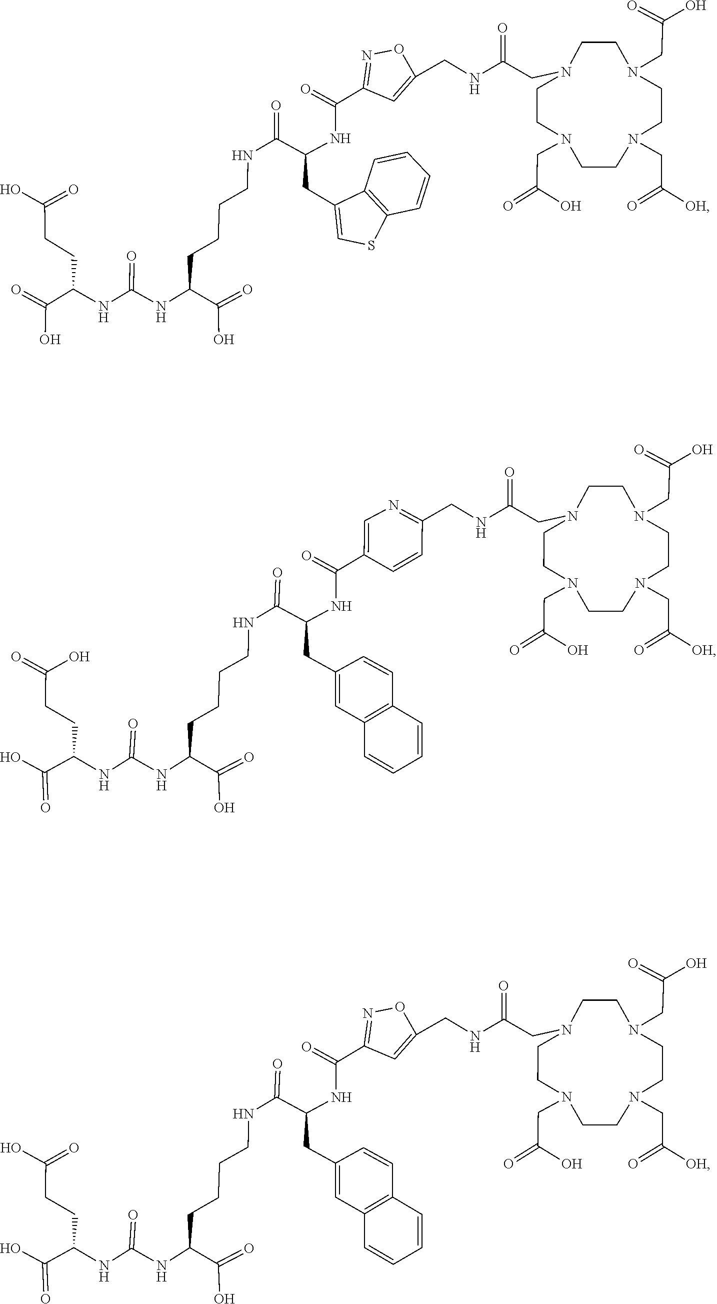 [SMILES: O=C(O)CC[C@H](NC(=O)N[C@@H](CCCCNC(=O)[C@H](Cc1csc2ccccc12)NC(=O)c1cc(CNC(=O)CN2CCN(CC(=O)O)CCN(CC(=O)O)CCN(CC(=O)O)CC2)on1)C(=O)O)C(=O)O ; O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)nc1)C(=O)O)C(=O)O ; O=C(O)CC[C@H](NC(=O)N[C@@H](CCCCNC(=O)[C@H](Cc1ccc2ccccc2c1)NC(=O)c1cc(CNC(=O)CN2CCN(CC(=O)O)CCN(CC(=O)O)CCN(CC(=O)O)CC2)on1)C(=O)O)C(=O)O]

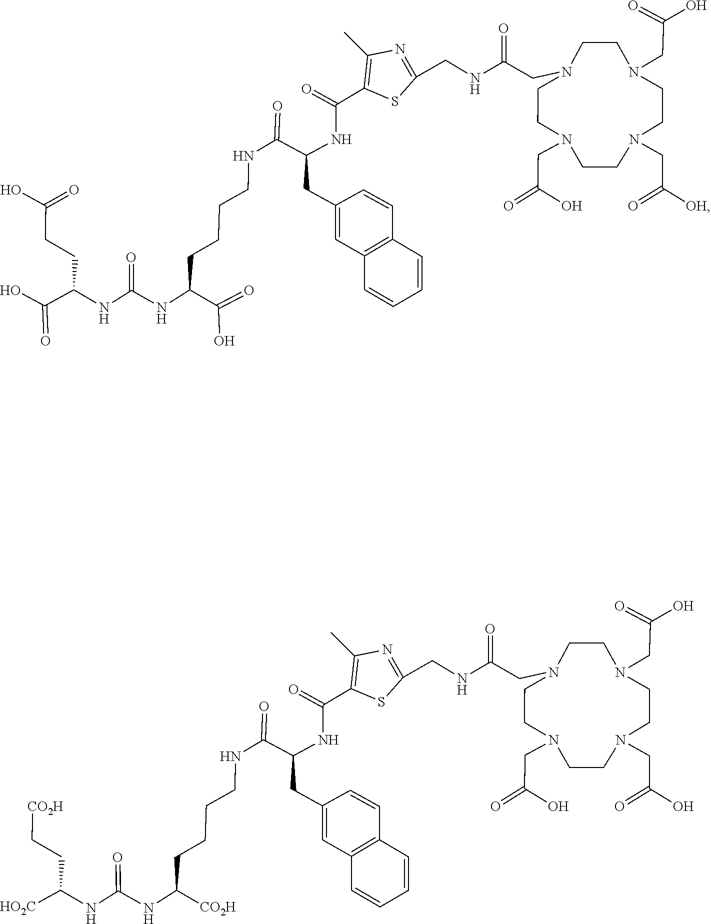 [SMILES: Cc1nc(CNC(=O)CN2CCN(CC(=O)O)CCN(CC(=O)O)CCN(CC(=O)O)CC2)sc1C(=O)N[C@@H](Cc1ccc2ccccc2c1)C(=O)NCCCC[C@H](NC(=O)N[C@@H](CCC(=O)O)C(=O)O)C(=O)O ; Cc1nc(CNC(=O)CN2CCN(CC(=O)O)CCN(CC(=O)O)CCN(CC(=O)O)CC2)sc1C(=O)N[C@@H](Cc1ccc2ccccc2c1)C(=O)NCCCC[C@H](NC(=O)N[C@@H](CCC(=O)O)C(=O)O)C(=O)O]

or a pharmaceutically acceptable salt thereof or a solvate thereof or a solvate of a salt thereof.

The compounds of the invention may, depending on their structure, exist in tautomeric or stereoisomeric forms (enantiomers, diastereomers). The invention therefore also encompasses the tautomers, enantiomers or diastereomers and respective mixtures thereof. The stereoisomerically uniform constituents can be isolated in a known manner from such mixtures of enantiomers and/or diastereomers.

In one embodiment the compounds of the present invention or a pharmaceutically acceptable salt thereof or a solvate thereof or a solvate of a salt thereof are used for the preparation of radiolabeled compounds.

In one embodiment, the subject-matter of the present invention is a metal complex comprising a radionuclide and a compound of any of the preceding embodiments or a pharmaceutically acceptable salt thereof or a solvate thereof or a solvate of a salt thereof. A metal complex can be formed by a complexation of a metal by a chelator. In one embodiment the radionuclide is selected from the group comprising ¹¹¹In, ⁹⁰Y, ⁶⁸Ga, ¹⁷⁷Lu, ^{99m}TC, ⁶⁴Cu, ⁶⁷Cu, ¹⁵³Gd, ¹⁵⁵Gd, ¹⁵⁷Gd, ²¹³Bi, ²²⁵Ac, ⁸⁹Zr, ²⁰³Pb, ²¹²Pb. In one embodiment the radionuclide is ¹⁷⁷Lu. In one embodiment the radionuclide is ⁶⁸Ga. In one embodiment the radionuclide is ²²⁵AC.

In a further aspect, the present invention relates to pharmaceutical and/or diagnostic compounds and pharmaceutically acceptable salts thereof or solvates thereof or salts of solvates thereof as well as pharmaceutical compositions or formulations comprising the same.

In one embodiment, the subject-matter of the present invention is a pharmaceutical composition comprising a metal complex of a compound of Formula I, or a pharmaceutically acceptable salt, or a solvate thereof, or a solvate of a salt thereof, and a pharmaceutically acceptable carrier. The pharmaceutical compositions according to the present invention may be administered by typical routes, e.g. orally or via a parenteral route, such as injection or infusion.

Corresponding to the kind of administration, compounds prepared according to the methods of the disclosure may be formulated into any suitable pharmaceutical formulation, e.g. in the form of solutions or suspensions, simple tablets or dragees, hard or soft gelatin capsules, suppositories, ovules, or preparations for injection, which are prepared according to common galenic methods.

In one embodiment, the subject-matter of the present invention is a metal complex of a compound of Formula I, or a pharmaceutically acceptable salt, or a solvate thereof, or a solvate of a salt thereof for use in a method of imaging in a patient.

In one embodiment, the subject-matter of the present invention is a metal complex of a compound of Formula I, or a pharmaceutically acceptable salt, or a solvate thereof, or a solvate of a salt thereof for use in a method of diagnosing cancer and/or metastasis thereof, optionally selected from the group of cancers that are positive for expression of PSMA, further optionally prostate cancer.

In one embodiment, the subject-matter of the present invention is a metal complex of a compound of Formula I, or a pharmaceutically acceptable salt, or a solvate thereof, or a solvate of a salt thereof for use in a method of treating cancer and/or metastasis thereof, optionally selected from the group of cancers that are positive for expression of PSMA, further optionally prostate cancer.

In one embodiment, the subject-matter of the present invention is a pharmaceutical composition according to the invention for use in a method of imaging in a patient. The concentration of the imaging agent or the therapeutic agent in the radiological vehicle should be sufficient to provide satisfactory imaging. For example, when using an aqueous solution, the dosage is about 1.0 to 100 millicuries. The actual dose administered to a patient for imaging or therapeutic purposes, however, is determined by the physician administering treatment.

In one embodiment, the subject-matter of the present invention is a pharmaceutical composition according to the invention for use in a method of diagnosing cancer and/or metastasis thereof, optionally selected from the group of cancers that are positive for expression of PSMA, further optionally prostate cancer.

In one embodiment, the subject-matter of the present invention is a pharmaceutical composition according to the invention for use in a method of treating cancer and/or metastasis thereof, optionally selected from the group of cancers that are positive for expression of PSMA, further optionally prostate cancer.

In one embodiment, the subject-matter of the present invention is a compound of Formula I, or a metal complex of a compound of Formula I, or a salt thereof, a solvate thereof, or a salt of a solvate thereof for manufacturing of a medicament for diagnosing or treating cancer and/or metastasis thereof, optionally selected from the group of cancers that are positive for expression of PSMA, further optionally prostate cancer.

In yet other aspects, the present application relates to methods of treatment of diseases or conditions and methods of diagnosis of such diseases or conditions with the herein provided PSMA-binding compounds comprising a structure as shown in Formula (I). The diseases or conditions are characterized by a statistically significant expression of PSMA, e.g. prostate cancer.

In one embodiment, the subject-matter of the present invention is a method of imaging in a patient, comprising administering to an individual a metal complex of a compound of Formula I, or a salt thereof, a solvate thereof, or a salt of a solvate thereof, or a pharmaceutical composition comprising a compound of Formula I, or a metal complex of a compound of Formula I, or a salt thereof, a solvate thereof, or a salt of a solvate thereof.

Imaging may be carried out in the normal manner, for example by injecting a sufficient amount of the imaging composition to provide adequate imaging and then scanning with a suitable imaging or scanning machine, such as a tomograph or gamma camera.

In one embodiment, the subject-matter of the present invention is a method of diagnosing cancer and/or metastasis thereof, optionally selected from the group of cancers that are positive for expression of PSMA, further optionally prostate cancer, comprising administering to an individual a metal complex of a compound of Formula I, or a salt thereof, a solvate thereof, or a salt of a solvate thereof, or a pharmaceutical composition comprising a metal complex of a compound of Formula I, or a salt thereof, a solvate thereof, or a salt of a solvate thereof.

In one embodiment, the subject-matter of the present invention is a method of treating cancer and/or metastasis thereof, optionally selected from the group of cancers that are positive for expression of PSMA, further optionally prostate cancer, comprising administering to an individual a metal complex of a compound of Formula I, or a salt thereof, a solvate thereof, or a salt of a solvate thereof, or a pharmaceutical composition comprising a metal complex of a compound of Formula I, or a salt thereof, a solvate thereof, or a salt of a solvate thereof.

With the above context, the following consecutively numbered embodiments provide further specific aspects of the invention:
1. A compound of Formula I

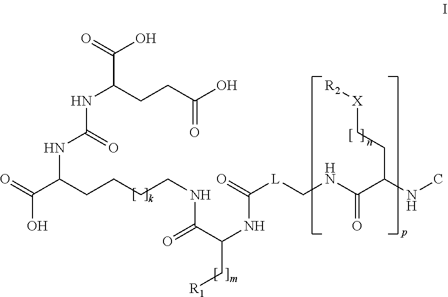

wherein
C is represented by any of the structures selected from the group consisting of
1,4,7,10-tetraazacyclododecane-1,4,7,10-tetraacetic acid (DOTA),
N,N″-bis[2-hydroxy-5-(carboxyethyl)benzyl]ethylenediamine-N,N″-diacetic acid (HBED-CC),
1,4-bis(carboxymethyl)-6-[bis(carboxymethyl)]amino-6-methylperhydro-1,4-diazepine (AAZTA),
1,4,7-triazacyclononane-1,4,7-triacetic acid (NOTA),
2-(4,7,10-tris(carboxymethyl)-1,4,7,10-tetraazacyclododecan-1-yl)pentanedioic acid (DOTAGA),
N⁴-[5-[[4-[[5-(acetylhydroxyamino)pentyl]amino]-1,4-dioxobutyl]hydroxyamino]pentyl]-N¹-(5-aminopentyl)-N¹-hydroxybutanediamide (DFO),
N¹-[5-(acetylhydroxyamino)pentyl]-N²⁶-(5-aminopentyl)-N²⁶,5,16-trihydroxy-4,12,15,23-tetraoxo-5,11,16,22-tetraazahexacosanediamide (DFO*), or
5-[(2-mercapto-2-methylpropyl)[2-[(2-mercapto-2-methylpropyl)amino]ethyl]amino]pentanoic acid (BAT),
   L is C1-C5-heteroaryl, optionally substituted with C1-C6 alkyl,
   X is NH or CH₂,
   k is 0, 1 or 2,
   m is an integer from 1 to 7,
   n is an integer from 0 to 6,
   p is 0 or 1,
   R₁ is phenyl, phenyl-NH—, benzothiophenyl or naphthyl,
   wherein phenyl and phenyl-NH— is optionally substituted with 1, 2 or 3 groups each independently selected from halo, OH and NO₂, and
   R₂ is aryl, aryl-C(O)—, or heteroaryl-C(O)—, optionally substituted with 1, 2 or 3 groups each independently selected from halo, OH, C1-C6-alkyl, OCH₃ and NO₂,
   wherein C1-C5-heteroaryl contains 1, 2, 3 or 4 heteroatoms, each independently selected from N, O or S,
   or a pharmaceutically acceptable salt thereof or a solvate thereof or a solvate of a salt thereof.
   2. The compound of Formula I according to embodiment 1 that is a compound of Formula II

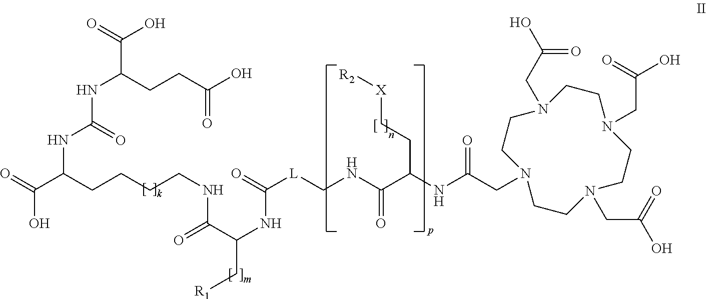

wherein
L, X, k, m, n, p, R₁ and R₂ are as defined in embodiment 1,
or a pharmaceutically acceptable salt thereof or a solvate thereof or a solvate of a salt thereof.
3. The compound of Formula I according to embodiment 1

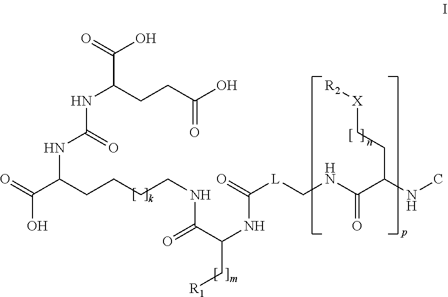

wherein
C, X, k, m, n, p, R₁ and R₂ are as defined in embodiment 1,
L is selected from the group consisting of isoxazolyl, pyridyl, thiazolyl and thiophenyl, optionally substituted with C1-C6 alkyl,
or a pharmaceutically acceptable salt thereof or a solvate thereof or a solvate of a salt thereof.
4. The compound of Formula I according to any one of embodiments 1 to 3 that is a compound of Formula III

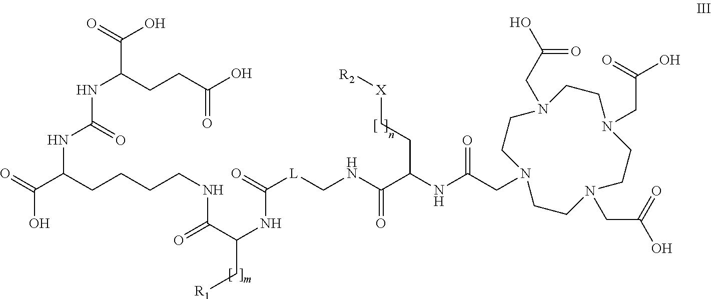

wherein
X, m, n, R₁ and R₂ are as defined in embodiment 1,
L is selected from the group consisting of isoxazolyl, pyridyl, thiazolyl and thiophenyl,
optionally substituted with C1-C6 alkyl,
or a pharmaceutically acceptable salt thereof or a solvate thereof or a solvate of a salt thereof.
5. The compound of Formula I according to any one of embodiments 1 to 3 that is a compound of Formula IV

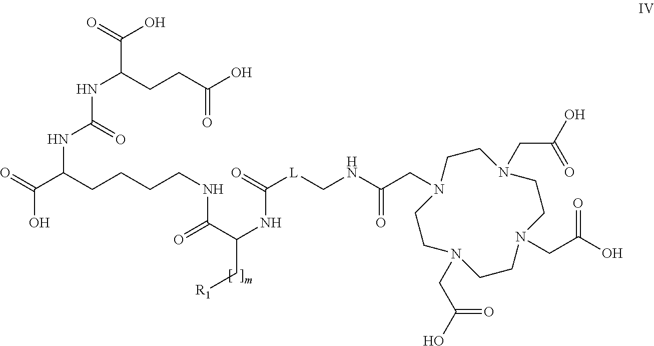

wherein
m and R₁ are as defined in embodiment 1,
L is selected from the group consisting of isoxazolyl, pyridyl, thiazolyl and thiophenyl, optionally substituted with C1-C6 alkyl,
or a pharmaceutically acceptable salt thereof or a solvate thereof or a solvate of a salt thereof.
6. The compound of Formula I according to any one of embodiments 1 to 4 that is a compound of Formula V

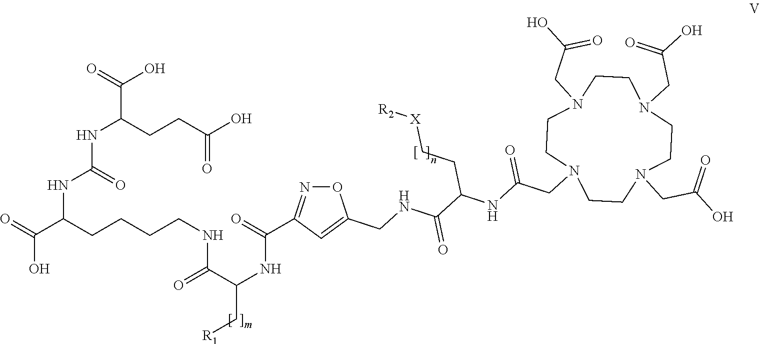

wherein
X, m, n, R₁ and R₂ are as defined in embodiment 1,
or a pharmaceutically acceptable salt thereof or a solvate thereof or a solvate of a salt thereof.
7. The compound of Formula I according to any one of embodiments 1 to 3 or 5 that is a compound of Formula VI

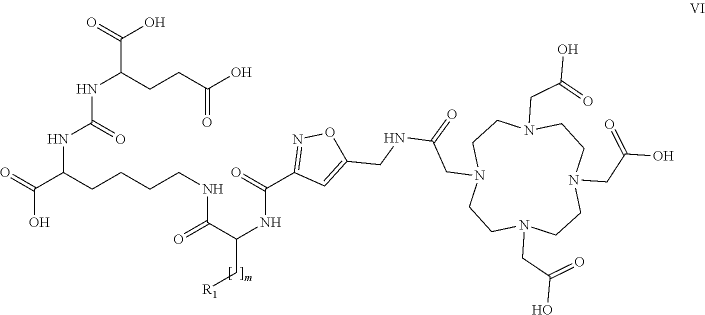

wherein
m and R₁ are as defined in embodiment 1,
or a pharmaceutically acceptable salt thereof or a solvate thereof or a solvate of a salt thereof.
8. The compound according to any one of embodiments 1 to 7, wherein R₁ is benzothiophenyl or naphthyl and m is 1, or a pharmaceutically acceptable salt thereof or a solvate thereof or a solvate of a salt thereof.
9. The compound according to any one of embodiments 1 to 3, that is a compound selected from the list of the following compounds:

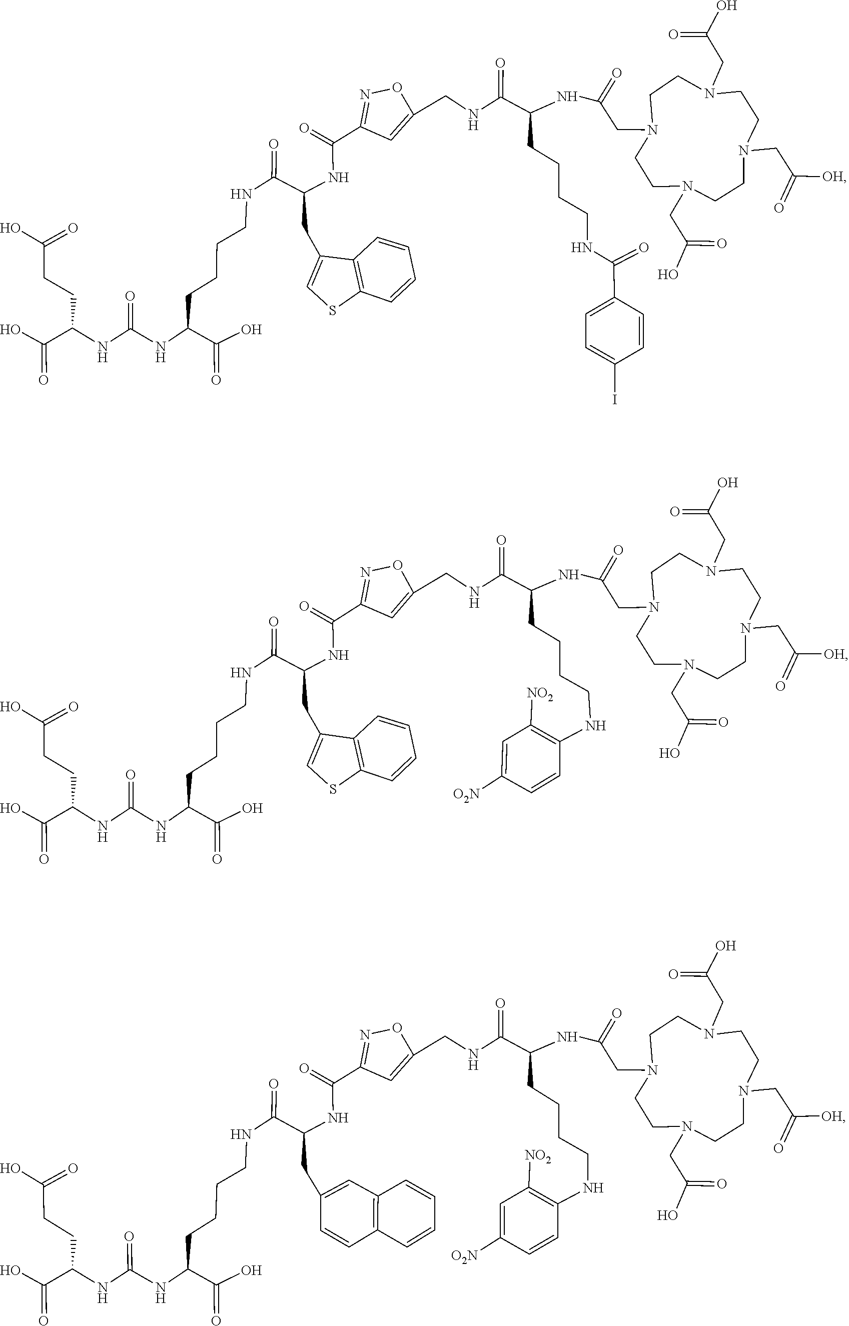

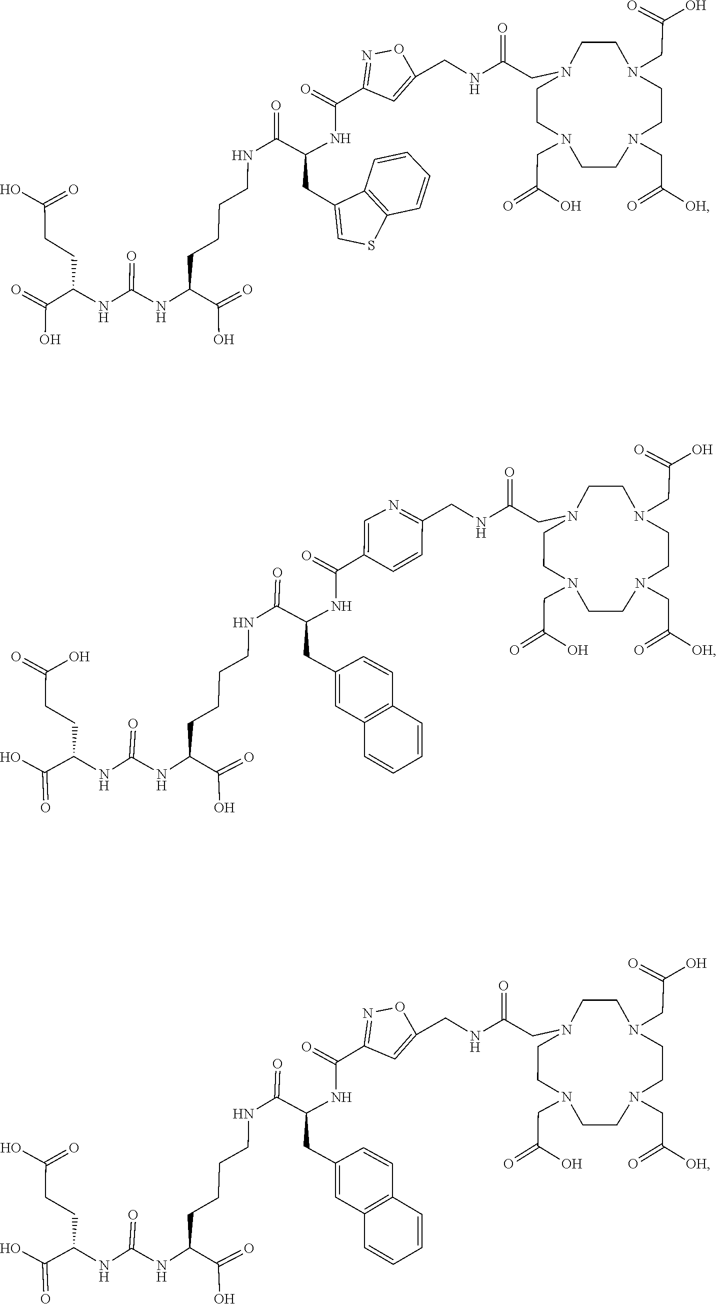 [SMILES: O=C(O)CC[C@H](NC(=O)N[C@@H](CCCCNC(=O)[C@H](Cc1csc2ccccc12)NC(=O)c1cc(CNC(=O)CN2CCN(CC(=O)O)CCN(CC(=O)O)CCN(CC(=O)O)CC2)on1)C(=O)O)C(=O)O ; O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)nc1)C(=O)O)C(=O)O ; O=C(O)CC[C@H](NC(=O)N[C@@H](CCCCNC(=O)[C@H](Cc1ccc2ccccc2c1)NC(=O)c1cc(CNC(=O)CN2CCN(CC(=O)O)CCN(CC(=O)O)CCN(CC(=O)O)CC2)on1)C(=O)O)C(=O)O]

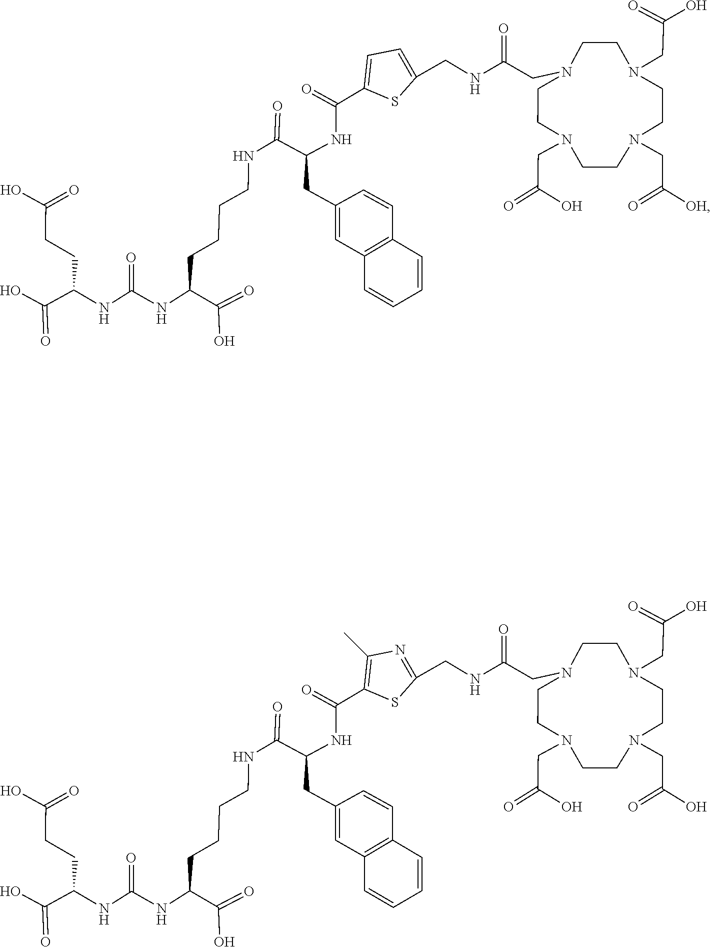 [SMILES: O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)s1)C(=O)O)C(=O)O ; Cc1nc(CNC(=O)CN2CCN(CC(=O)O)CCN(CC(=O)O)CCN(CC(=O)O)CC2)sc1C(=O)N[C@@H](Cc1ccc2ccccc2c1)C(=O)NCCCC[C@H](NC(=O)N[C@@H](CCC(=O)O)C(=O)O)C(=O)O]

or a pharmaceutically acceptable salt thereof or a solvate thereof or a solvate of a salt thereof.
10. Use of a compound according to any one of embodiments 1 to 9 or a pharmaceutically acceptable salt thereof or a solvate thereof or a solvate of a salt thereof for the preparation of radiolabeled compounds.
11. A metal complex comprising a radionuclide and a compound of any one of embodiments 1 to 9 or a pharmaceutically acceptable salt thereof or a solvate thereof or a solvate of a salt thereof.
12. The metal complex according to embodiment 11, wherein the radionuclide is ¹¹¹In, ⁹⁰Y, ⁶⁸Ga, ¹⁷⁷Lu, ^{99m}Tc, ⁶⁴Cu, ⁶⁷Cu, ¹⁵³Gd, ¹⁵⁵Gd , ¹⁵⁷Gd , ²¹³Bi, ²²⁵Ac, ⁸⁹Zr, ²⁰³Pb, ²¹²Pb.
13. The metal complex according to embodiment 11 or 12, wherein the radionuclide is ¹⁷⁷Lu.
14. The metal complex according to embodiment 11 or 12, wherein the radionuclide is ⁶⁸Ga.
15. The metal complex according to embodiment 11 or 12, wherein the radionuclide is ²²⁵Ac.
16. A pharmaceutical composition comprising a compound of any one of embodiments 1 to 9 or a metal complex according to any one of embodiments 11 to 15, or a pharmaceutically acceptable salt, or a solvate thereof, or a solvate of a salt thereof, and a pharmaceutically acceptable carrier.
17. A metal complex according to any one of embodiments 11 to 15 or a pharmaceutically acceptable salt thereof or a solvate thereof or a solvate of a salt thereof for use in a method of imaging in a patient.
18. A metal complex according to any one of embodiments 11 to 15 or a pharmaceutically acceptable salt thereof or a solvate thereof or a solvate of a salt thereof for use in a method of diagnosing a cancer and/or metastasis thereof, optionally selected from the group of cancers that are positive for expression of PSMA, further optionally prostate cancer.
19. A metal complex according to any one of embodiments 11 to 15 or a pharmaceutically acceptable salt thereof or a solvate thereof or a solvate of a salt thereof for use in a method for treating cancer and/or metastasis thereof, optionally selected from the group of cancers that are positive for expression of PSMA, further optionally prostate cancer.
20. A method of diagnosing cancer and/or metastasis thereof, optionally selected from the group of cancers that are positive for expression of PSMA, further optionally prostate cancer, comprising administering to an individual a metal complex according to any one of embodiments 11 to 15 or a pharmaceutically acceptable salt thereof or a solvate thereof or a solvate of a salt thereof.
21. A method of treating cancer and/or metastasis thereof, optionally selected from the group of cancers that are positive for expression of PSMA, further optionally prostate cancer, comprising administering to an individual a metal complex according to any one of embodiments 11 to 15 or a pharmaceutically acceptable salt thereof or a solvate thereof or a solvate of a salt thereof.
22. A compound according to any one of embodiments 1 to 8, with the proviso that when p is 0 and L is isoxazolyl, R₁ is not naphthyl.

### DEFINITIONS

Listed below are definitions of various terms used herein. These definitions apply to the terms as they are used throughout this specification and claims unless otherwise limited in specific instances either individually or as part of a larger group.

Unless defined otherwise, all technical and scientific terms used herein generally have the same meaning as commonly understood by one of ordinary skill in the art. Generally the nomenclature used herein and the synthetic procedures, organic chemistry, and peptide chemistry are those well-known and commonly employed in the art.

As used herein the articles “a” and “an” refer to one or to more than one (i.e. to at least one) of the grammatical object of the article. By way of example, “an element” means one element or more than one element. Furthermore, use of the term “including,” as well as other forms such as “include,” “includes,” and “included,” is intended to be open and not limiting.

As used herein, the term “alkyl” by itself or as part of another substituent refers to a straight or branched chain hydrocarbon having from 1 to 16 carbon atoms (C1-C16 alkyl), preferably from 1 to 12 carbon atoms (C1-C12-alkyl), or more preferably from 1 to 6 carbon atoms (C1-C6-alkyl). Examples include, but not limited to: methyl, ethyl, propyl, isopropyl, butyl, isobutyl, tert-butyl, pentyl, neopentyl, and hexyl.

As used herein the term “cycloalkyl” refers to a monocyclic or polycyclic nonaromatic group wherein each of the atoms forming the ring (i.e. skeletal atoms) is a carbon atom. In one embodiment, the cycloalkyl group is saturated or partially unsaturated. Cycloalkyl groups include groups having 3 to 10 ring atoms (C3-C10-cycloalkyl), groups having 3 to 8 ring atoms (C3-C8-cycloalkyl), groups having 3 to 7 ring atoms (C3-C7-cycloalkyl), groups having 3 to 6 ring atoms (C3-C6-cycloalkyl) and groups having 5 or 6 ring atoms (C5-C6-cycloalkyl). Examples of cycloalkyls include but are not limited to cyclopropyl, cyclobutyl, cyclopentyl, cyclohexyl, cycloheptyl and cyclooctyl.

As used herein the term “aryl” employed alone or in combination with other terms, means unless otherwise stated a carbocyclic aromatic system containing one or more rings (typically one, two or three rings) wherein such rings may be attached together in a pendant manner such as a biphenyl, or may be fused, such as naphthalene. Examples of aryl groups include phenyl, anthracyl, and naphthyl. In some embodiments aryl groups have from six to sixteen carbon atoms. In some embodiments aryl groups have from six to twelve carbon atoms (e.g. C6-C12-aryl). In some embodiments, aryl groups have six carbon atoms (e.g. C6-aryl).

As used herein the terms “heteroaryl” refers to a heterocycle having aromatic character containing one or more rings (typically one, two or three rings), that contains one to four ring heteroatoms each independently selected from oxygen, sulfur and nitrogen. Heteroaryl substituents may be defined by the number of carbon atoms e.g. C1-C5-heteroaryl indicates the number of carbon atoms contained in the heteroaryl group without including the number of heteroatoms. For example a C1-C5-heteroaryl will include an additional one to four heteroatoms. Examples of heteoaryl include but are not limited to pyridyl, pyrazinyl , pyrimidinyl (including e.g. 2-and 4-pyrimidinyl), pyridazinyl, thiothienylfuryl, pyrrolyl (including e.g. 2-pyrrolyl), imidazolyl, thiazolyl, thiophenyl, isoxazolyl, oxazolyl, pyrazolyl (including e.g. 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl , 1,3,4-thiadiazolyland, 1,3,4-oxadiazolyl. Preferably heteroaryl is isoxazolyl, pyridyl, thiazolyl or thiophenyl.

As used herein, the term “halo” or “halogen” alone or as part of another substituent means unless otherwise stated a fluorine, chlorine, bromine, or iodine atom. For the avoidance of doubt, where two halo moieties are present in a group, they may be the same or different.

As used herein the term “pharmaceutically acceptable” refers to a material such as a carrier or diluent which does not abrogate the biological activity or properties of the compound and is relatively non-toxic i.e. the material may be administered to an individual without causing undesirable biological effects or interacting in a deleterious manner with any of the components of the composition in which it is contained.

As used herein the term “a salt” refers to derivatives of the disclosed compounds wherein the parent compound is modified by converting an existing acid or base moiety to its salt form. Examples of pharmaceutically acceptable salts include but are not limited to mineral or organic acid salts of basic residues such as amines; alkali or organic salts of acidic residues such as carboxylic acids; and the like. The pharmaceutically acceptable salts of the present invention include the conventional non-toxic salts of the parent compound formed for examplefrom non-toxic inorganic or organic acids. Pharmaceutically acceptable salts of the compounds according to the invention include acid addition salts, for example, but not limited to, salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid. Pharmaceutically acceptable salts of the compounds according to the invention also include salts of customary bases, for example, but not limited to, alkali metal salts (for example sodium and potassium salts), alkaline earth metal salts (for example calcium and magnesium salts) and ammonium salts derived from ammonia or organic amines having 1 to 16 carbon atoms, such as, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

As used herein, the term “solvate” refers to compounds which form a complex in the solid or liquid state by coordination with solvent molecules. Suitable solvents include, but are not limited to, methanol, ethanol, acetic acid and water. Hydrates are a special form of solvates in which the coordination takes place with water.

As used herein the term “composition” or “pharmaceutical composition” refers to a mixture of at least one compound according to the invention with a pharmaceutically acceptable carrier. The pharmaceutical composition facilitates administration of the compound to a patient or subject. Multiple techniques of administering a compound exist in the art including but not limited to intravenous, oral, aerosol, rectal, parenteral, ophthalmic, pulmonary and topical administration.

As used herein the term “pharmaceutically acceptable carrier” means a pharmaceutically acceptable material, composition or carrier such as a liquid or solid filler, stabilizer, dispersing agent, suspending agent, diluent, excipient, thickening agent , solvent or encapsulating material involved in carrying or transporting a compound useful within the invention within or to the patient such that it may perform its intended function.

As used herein, the terms “treat,” “treatment,” or “treating” refer to the application or administration of a therapeutic agent, e.g., a compound of the disclosure (alone or in combination with another pharmaceutical agent) to a patient, or application or administration of a therapeutic agent to an isolated tissue or cell line from a patient (e.g. for diagnosis or ex vivo applications) who has cancer, a symptom of cancer, or the potential to develop cancer with the purpose to cure, heal, alleviate, relieve, alter, remedy, ameliorate, improve, or affect cancer, the symptoms of cancer, or the potential to develop cancer.

As used herein, the terms “patient,” “individual,” or “subject” refer to a human or a non-human mammal. Non-human mammals include, for example, livestock and pets such as ovine, bovine, porcine, feline, and murine mammals. Preferably the patient, subject, or individual is human.

As used herein, the terms “diagnose,” “diagnosing,” or “diagnosis” refer to methods by which the skilled medical practitioner can estimate or determine whether or not a subject is suffering from a given disease or condition.

As used herein, the phrase “independently selected from the group consisting of” is understood to indicate that each instance of a variable (e.g. a substituent in a particular formula) may be independently chosen from other instances of the same variable (e.g. other instances of the same substituent in the same formula), and that different variables may be chosen independent of each other.

DOTA is an abbreviation of the chelator 1,4,7,10-tetraazacyclododecane-1,4,7,10-tetraacetic acid.

HBED-CC is an abbreviation of the chelator N,N″-bis[2-hydroxy-5-(carboxyethyl)benzyl]ethylenediamine-N,N″-diacetic acid.

AAZTA is an abbreviation of the chelator 1,4-bis(carboxymethyl)-6-[bis(carboxymethyl)]amino-6-methylperhydro-1,4-diazepine.

NOTA is an abbreviation of the chelator 1,4,7-triazacyclononane-1,4,7-triacetic acid.

DOTAGA is an abbreviation of the chelator 2-(4,7,10-tris(carboxymethyl)-1,4,7,10-tetraazacyclododecan-1-yl)pentanedioic acid.

DFO is an abbreviation of the chelator N⁴-[5-[[4-[[5-(acetylhydroxyamino)pentyl]amino]-1,4-dioxobutyl]hydroxyam ino]pentyl]-N¹-(5-aminopentyl)-N¹-hydroxybutanediamide (CAS No. 70-51-9).

DFO* is an abbreviation of the chelator N¹-[5-(acetylhydroxyamino)pentyl]-N² -(5-aminopentyl)-N²⁶,5,16-trihydroxy-4,12,15,23-tetraoxo-5,11,16,22-tetraazahexacosanediamide (CAS No. 1623757-38-9).

BAT is an abbreviation of the chelator 5-[(2-mercapto-2-methylpropyl)[2-[(2-mercapto-2-methylpropyl)amino]ethyl]amino]pentanoic acid (CAS No. 445311-88-6).

DOTA-NHS ester is an abbreviation of the chemical compound 2,2′,2″-(10-(2-((2,5-dioxopyrrolidin-1-yl)oxy)-2-oxoethyl)-1,4,7,10-tetraazacyclododecane-1,4,7-triyl)triacetic acid.

As used herein the term “PSMA-617” refers to the chemical compound (3S,10S,14S)-3-[(naphthalen-2-yl)methyl]-1,4,12-trioxo-1-[(1R,4S)-4-[[2-[4,7,10-tris(carboxymethyl)-1,4,7,10-etraazacyclododecan-1- yl]acetamido]methyl]cyclo-hexyl]-2,5,11 ,13-tetraazahexadecane-10,14,16-tricarboxylic acid.

Fmoc is an abbreviation of the chemical group fluorenylmethyloxycarbonyl.

Dde is an abbreviation of the chemical group N-(1-(4,4-dimethyl-2,6-dioxocyclohexylidene)ethyl).

PyBOP is an abbreviation of the chemical compound (benzotriazol-1-yloxy)tripyrrolidinophosphonium hexafluorophosphate

HOBt is an abbreviation of the chemical compound hydroxybenzotriazole.

HBTU is an abbreviation of the chemical compound O-(1H-benzotriazol-1-yl)-1,1,3,3-tetramethyluroniumhexafluorophosphate.

Lys is an abbreviation of the amino acid lysine.

DMF is an abbreviation of the chemical compound dimethylformamide.

TFA is an abbreviation of the chemical compound trifluoroacetic acid.

TEA is an abbreviation of the chemical compound trimethylamine.

DIPEA is an abbreviation of the chemical compound N,N-diisopropylethylamine

### DESCRIPTION OF THE FIGURES

FIG. 1 shows the general scheme of the synthesis of DOTA conjugated-PSMA ligands of the invention. The signification of the abbreviations used in FIG. 1 is as follows: TFA=Trifluoro acetic acid; CDI=1,1′-Carbonyldiimidazole; DCM=Dichloromethane; DMF=N,N-Dimethylformamide; IPA=2-Propanole; NMM=4-Methylmorpholine; coupling reagent=COMU or PyBOP, base=TEA or DIPEA

FIGS. 2-9 show the results of μPET-Imaging for each of the tested compounds at 20-40 minutes (A), 40-60 minutes (B) and 120-140 minutes (C). FIG. 2A-2C: PSMA617. FIG. 3A-3C: ABX271; FIG. 4A-4C: ABX288; FIG. 5A-5C: ABX289; FIG. 6A-6C: ABX338; FIG. 7A-7C: ABX339; FIG. 8A-8C: ABX341; FIG. 9A-9C: ABX346.

### EXAMPLES

### Example 1: Synthesis of DOTA-Conjugated PSMA Ligands

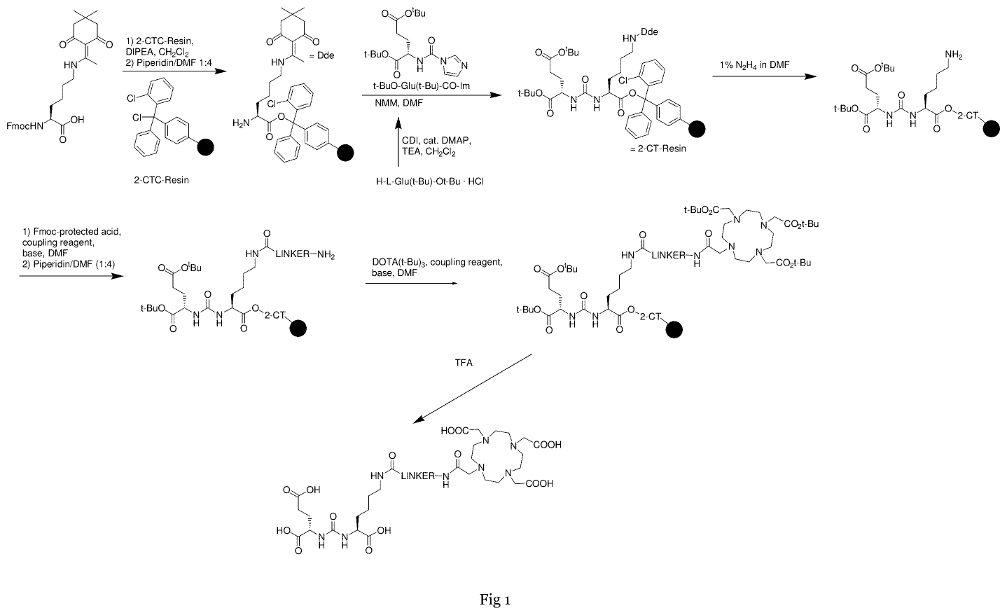

The DOTA-conjugated PSMA ligands were synthesized via solid-phase peptide synthesis (FIG. 1). In the first step, 0.3 mmol of Fmoc-Lys(Dde)-OH were immobilized on 2-chloro-trityl resin. After Fmoc-deprotection Di-tert-butyl (1H-imidazole-1-carbonyl)-L-glutamate (intermediate 1) was added and the mixture was reacted for 16 h with gentle agitation. The resin was filtered off and the Dde-protecting group was removed using 1% hydrazine hydrate in DMF according to standard Fmoc-deprotection procedures.

The subsequent synthesis of the peptidomimetic PSMA binding motif was performed according to standard Fmoc solid phase protocols. The consecutive coupling of each linker part was performed using 2 equivalents of the corresponding Fmoc-protected acid (FMPA1, FMPA2, FMPA3), 2 equivalents of PyBOP, 2 equivalents of HOBt and 3 equivalents of N-ethyl-diisopropylamine in DMF.

After activation with PyBOP, HOBt and DIPEA, 1.5 eq of tris(t-bu)-DOTA (Chematech) relative to the resin loading were coupled in DMF. The product was cleaved from the resin in a mixture consisting of trifluoroacetic acid, triisopropylsilane, and hydrochloric acid (95:2.5:2.5).

Alternatively the chelator was conjugated using HBTU activated DOTA-NHS ester (Chematech) or DOTA-TFP ester.

Analysis of the synthesized molecules was performed using reversed-phase high performance liquid chromatography (RP-HPLC; Ascentis Express C18, 150×4.6 mm; Supelco, Germany) with a linear A-B gradient (5% B to 100% B in 10 min) at a flow rate of 1.5 mL/min (analysis). Purification was performed using reversed-phase high performance liquid chromatography (RP-HPLC; Gemini-NX C18, 250×50 mm; Phenomenex, Germany) with a linear A-B gradient at a flow rate of 100 mL/min. Solvent A consisted of 0.1% aqueous TFA and solvent B was 0.1% TFA in CH₃CN.

The HPLC system (Dionex Ultimate 3000; Thermo-Fisher, Germany) was equipped with a UV detector. UV absorbance was measured at 200, 210 and 230 nm. Mass spectrometry was performed with a LC-MS (Dionex 3000, Thermo-Fisher, Germany).

### Example 2: Synthesis of Intermediate 1

Under argon atmosphere 25 g of bis(tert-butyl)-L-glutamate hydrochloride were disssolved in 250 ml of dichloromethane and cooled to 0° C. Then 0.414 g of 4-(Dimethylamino)pyridine and 29.5 ml of triethylamine were added and subsequently, 15 g of 1,1-carbonyldiimidazole were added in small portions. The reaction mixture was stirred overnight. An aqueous solution of NaHCO₃ was added and subsequently extracted with dichloromethane. Solvent was evaporated and the crude product was purified using column chromatography (dichloromethane/methanol, 80:1 (v/v)).

### Example 3: Synthesis of Compound ABX 346

In the first step, 0.3 mmol of Fmoc-Lys(Dde)-OH were immobilized on 2-chloro-tritylresin. After Fmoc-deprotection di-tert-butyl (1H-imidazole-1-carbonyl)-L-glutamate (intermediate 1) was added and the mixture was reacted for 16 h with gentle agitation. The resin was filtered off and the Dde-protecting group was removed using 1% hydrazine hydrate in DMF according to standard Fmoc-deprotection procedures. The consecutive coupling of Fmoc-3-benzothienylalanine (FMPA1), Fmoc-5-(Aminomethyl)-1,2-oxazole-3-carboxylic acid (FMPA2) and Fmoc-Lys(Dde)-OH (FMPA3) linker was performed using 2 equivalents of the corresponding Fmoc-protected acid (FMPA1, FMPA2, FMPA3), 2 equivalents of PyBOP, 2 equivalents of HOBt and 3 equivalents of N-ethyl-diisopropylamine in DMF. After the reaction, the resin was filtered off and the Dde-protecting group was removed using 1% hydrazine hydrate in DMF according to standard Fmoc-deprotection procedures. After Fmoc-deprotection 4-iodobezoic acid was coupled by using 2 equivalents of PyBOP, 2 equivalents of HOBt and 3 equivalents of N-ethyl-diisopropylamine and DMF.

After activation with PyBOP, HOBt and DIPEA, 1.5 eq of tris(t-bu)-DOTA (Chematech) relative to the resin loading were coupled in DMF. The product was cleaved from the resin in a mixture consisting of trifluoroacetic acid, triisopropylsilane, and hydrochloric acid (95:2.5:2.5).

Purification was performed using reversed-phase high performance liquid chromatography (RP-HPLC; Gemini-NX C18, 250×50 mm; Phenomenex, Germany) with a linear A-B gradient at a flow rate of 100 mL/min. Solvent A consisted of 0.1% aqueous TFA and solvent B was 0.1% TFA in CH₃CN.

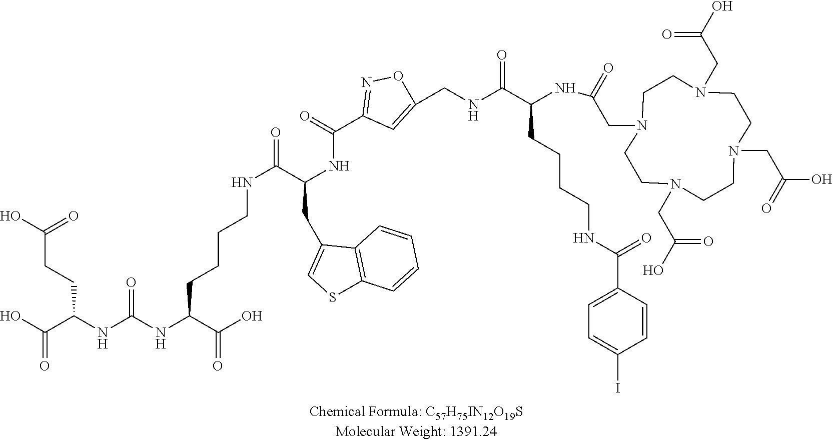 [SMILES: O=C(O)CC[C@H](NC(=O)N[C@@H](CCCCNC(=O)[C@H](Cc1csc2ccccc12)NC(=O)c1cc(CNC(=O)[C@H](CCCCNC(=O)c2ccc(I)cc2)NC(=O)CN2CCN(CC(=O)O)CCN(CC(=O)O)CCN(CC(=O)O)CC2)on1)C(=O)O)C(=O)O]

The compounds in the examples 4-10 have been prepared in a similar way.

### Example 4: ABX 341

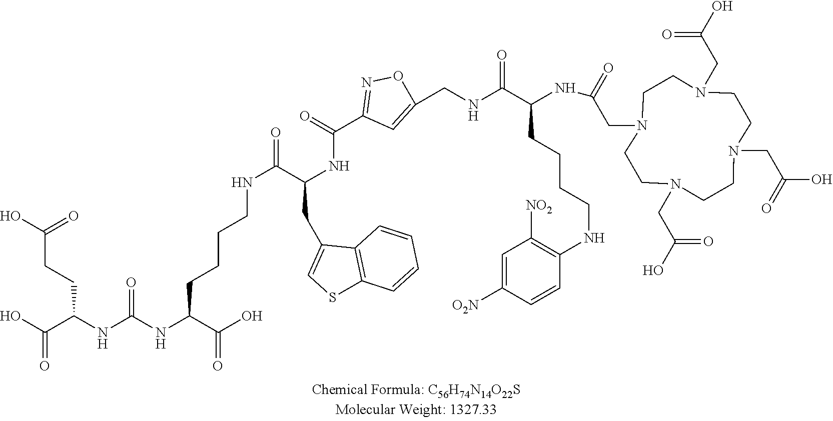 [SMILES: O=C(O)CC[C@H](NC(=O)N[C@@H](CCCCNC(=O)[C@H](Cc1csc2ccccc12)NC(=O)c1cc(CNC(=O)[C@H](CCCCNc2ccc([N+](=O)[O-])cc2[N+](=O)[O-])NC(=O)CN2CCN(CC(=O)O)CCN(CC(=O)O)CCN(CC(=O)O)CC2)on1)C(=O)O)C(=O)O]

### Example 5: ABX 339

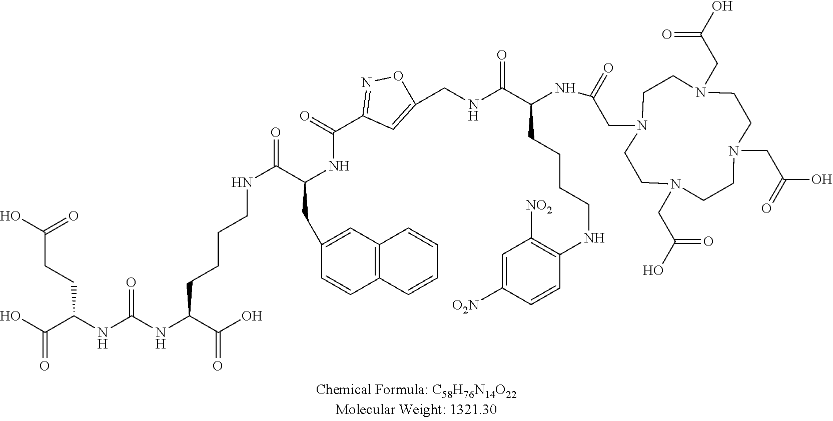 [SMILES: O=C(O)CC[C@H](NC(=O)N[C@@H](CCCCNC(=O)[C@H](Cc1ccc2ccccc2c1)NC(=O)c1cc(CNC(=O)[C@H](CCCCNc2ccc([N+](=O)[O-])cc2[N+](=O)[O-])NC(=O)CN2CCN(CC(=O)O)CCN(CC(=O)O)CCN(CC(=O)O)CC2)on1)C(=O)O)C(=O)O]

### Example 6: ABX 338

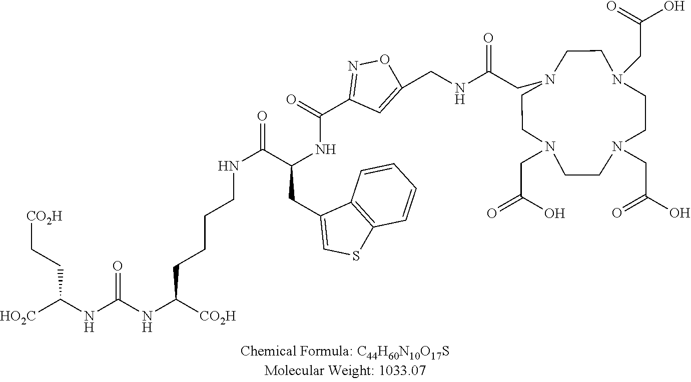 [SMILES: O=C(O)CC[C@H](NC(=O)N[C@@H](CCCCNC(=O)[C@H](Cc1csc2ccccc12)NC(=O)c1cc(CNC(=O)CN2CCN(CC(=O)O)CCN(CC(=O)O)CCN(CC(=O)O)CC2)on1)C(=O)O)C(=O)O]

### Example 7: ABX 289

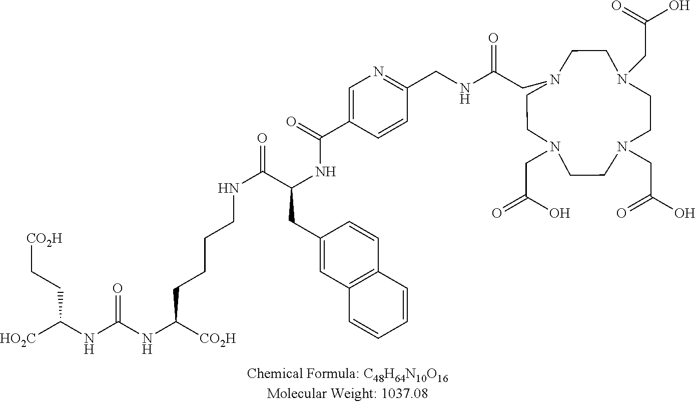 [SMILES: O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)nc1)C(=O)O)C(=O)O]

### Example 8: ABX 288

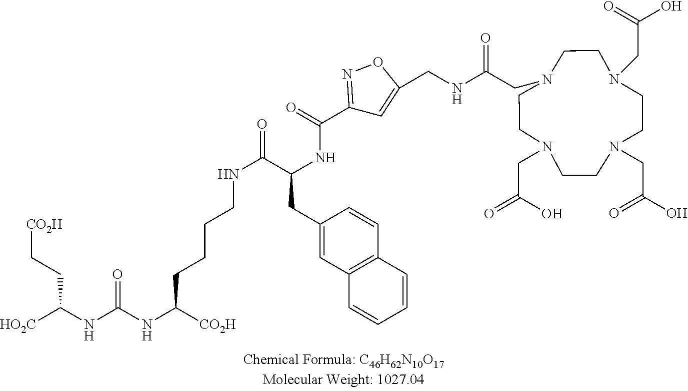 [SMILES: O=C(O)CC[C@H](NC(=O)N[C@@H](CCCCNC(=O)[C@H](Cc1ccc2ccccc2c1)NC(=O)c1cc(CNC(=O)CN2CCN(CC(=O)O)CCN(CC(=O)O)CCN(CC(=O)O)CC2)on1)C(=O)O)C(=O)O]

### Example 9: ABX 272

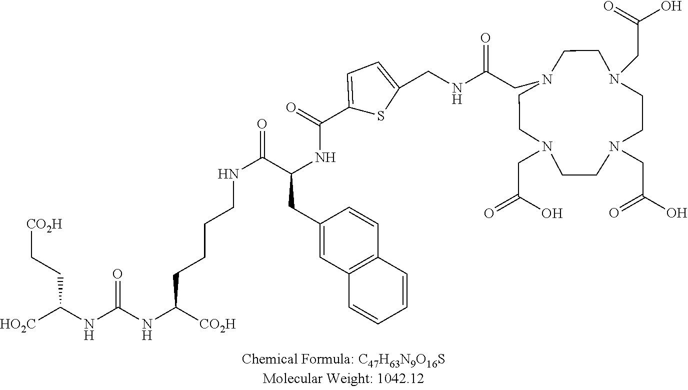 [SMILES: O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)s1)C(=O)O)C(=O)O]

### Example 10: ABX 271

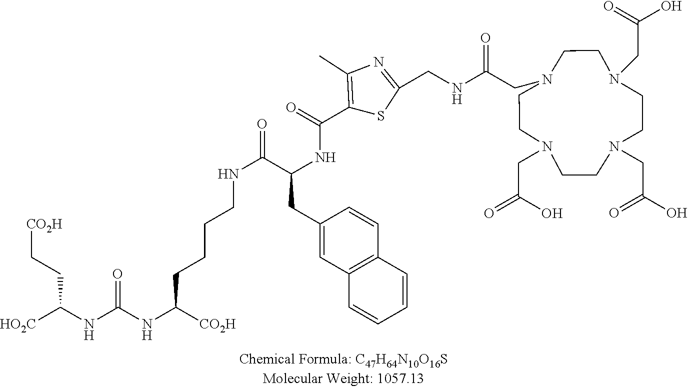 [SMILES: Cc1nc(CNC(=O)CN2CCN(CC(=O)O)CCN(CC(=O)O)CCN(CC(=O)O)CC2)sc1C(=O)N[C@@H](Cc1ccc2ccccc2c1)C(=O)NCCCC[C@H](NC(=O)N[C@@H](CCC(=O)O)C(=O)O)C(=O)O]

### Example 11: Radiolabeling

### Labeling With Ga-68

Gallium labeling was carried out using 90 μL HEPES in water (580 mg/mL; ultrapure-grade, Merck, Darmstadt, Germany) mixed with 40 μL of [⁶⁸Ga]GaCl₃. The pH of the mixture was adjusted to 3.8 to 4.1 with NaOH (30%; ultrapure-grade, Merck, Darmstadt, Germany). Subsequently 1.0 nmol of a synthesized compound of Example 3 to 10 was added and incubated at 98 ° C. for 10 to 15 minutes. For the competitive cell assay, the precursor amount was diminished to 0.5 nmol.

### Labeling With Lu-177

Lutetium labelling was carried out using 115 μL of sodium acetate buffer (400 mM, pH mixed with 10 to 20 MBq [177Lu]LuCl₃. Subsequently, 1.0 nmol of a synthesized compound of Example 3 to 10 was added and incubated at 98 ° C. for 20 to 25 minutes.

### Quality Control

The radiochemical purity of the labelled compounds was evaluated by analytical RP-HPLC (Reversed Phase High Performance Liquid Chromatography, Chromolith RP-18e, Merck, Darmstadt, Germany) using a linear gradient from 100% water to 100% acetonitrile in 5 minutes at a flow rate of 4 ml/min. The purity was evaluated by RP-TLC (Reversed Phase Thin Layer Chromatography) using 0.1 M citrate buffer as the mobile phase.

### Example 12: Determination of the Competitive Binding Affinity

Cell binding studies were performed using human PSMA+ LNCaP cells (ATCC CRL-1740) cultured in RPMI 1640 medium supplemented with 10% fetal bovine serum (Sera plus, PAN Biotech) and 2 mmol/l stable glutamine (PAN Biotech). Cells were grown at 37° C. in an incubator with humidified air and 5% CO₂. Trypsin/EDTA 0.5%/0.02% in PBS (PAN Biotech) was used to harvest cells.

### Internalization

1×10⁵ LNCaP cells were seeded in poly-L-lysine coated 24-well-plates for 24 h at 37° C. humidified air with 5% CO₂. After removing the medium, cells were incubated with radiolabelled compounds of Example 11 (30 nM) for 45 minutes at 37° C. and at 4° C. respectively. For determination of specific cell uptake, cells were blocked with PMPA (Tocris) at a concentration of 500 μM. The cells were washed three times with 1 ml ice-cold PBS, twice with 500 μl glycine-HCl (50mM; pH 2.8) for 5 min at rt to remove the surface-bound radioactivity and with 1 ml ice-cold PBS. Subsequent the cells were lysed with 0.5 ml 0.3M NaOH. The internalized and the surface-bound fractions were measured in a gammacounter.

The results are shown in table 1.

TABLE 1
Internalisation rate of PSMA binding compounds

| compound | internalisation rate (%) |
|---|---|
| PSMA-617 | 31.1 |
| EP2862857-MB17 | |
| Example ABX346 | 46.7 |
| Example ABX341 | 61.3 |
| Example ABX339 | 60.6 |
| Example ABX338 | 40.8 |
| Example ABX289 | 55.7 |
| Example ABX288 | 33.2 |
| Example ABX272 | 41.4 |
| Example ABX271 | 37.8 |

As can be seen from Table 1, the PSMA-binding ligands of the present invention which contain heteroaromatic linker building blocks show significantly higher internalisation rates than compound PSMA-617 known from the prior art.

### μPET-Imaging

5×10^{b 6} LNCAP cells in Opti-MEM 50% Matrigel (Corning) were inoculated subcutaneously into the trunk of male balbc nu/nu mice (Charles River). The tumors were allowed to grow until approximately 1 cm³ in size.

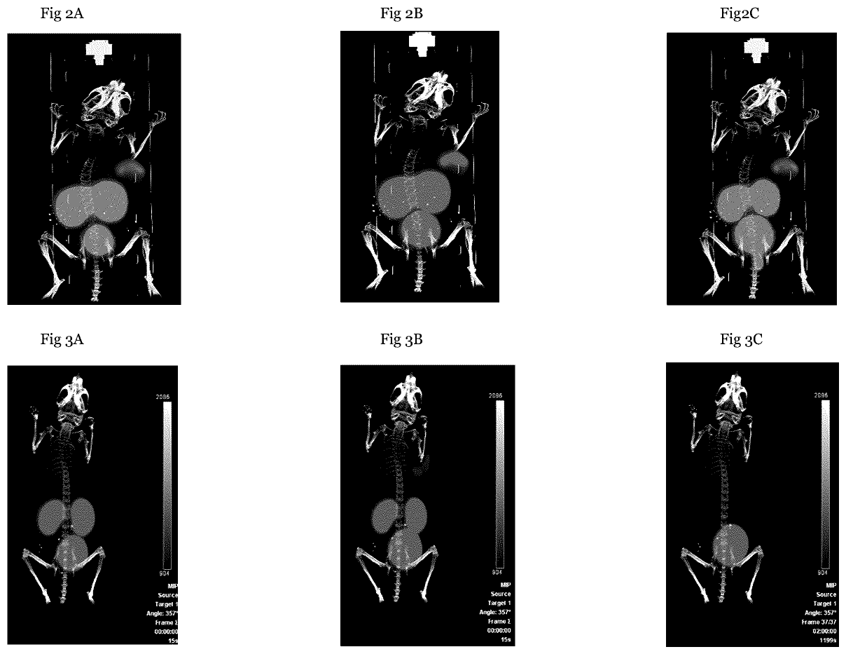

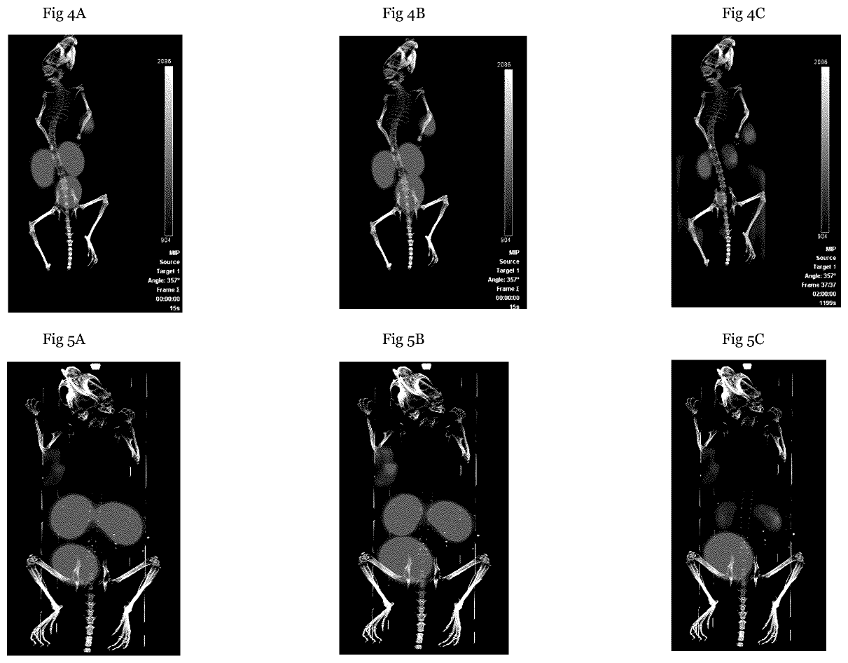

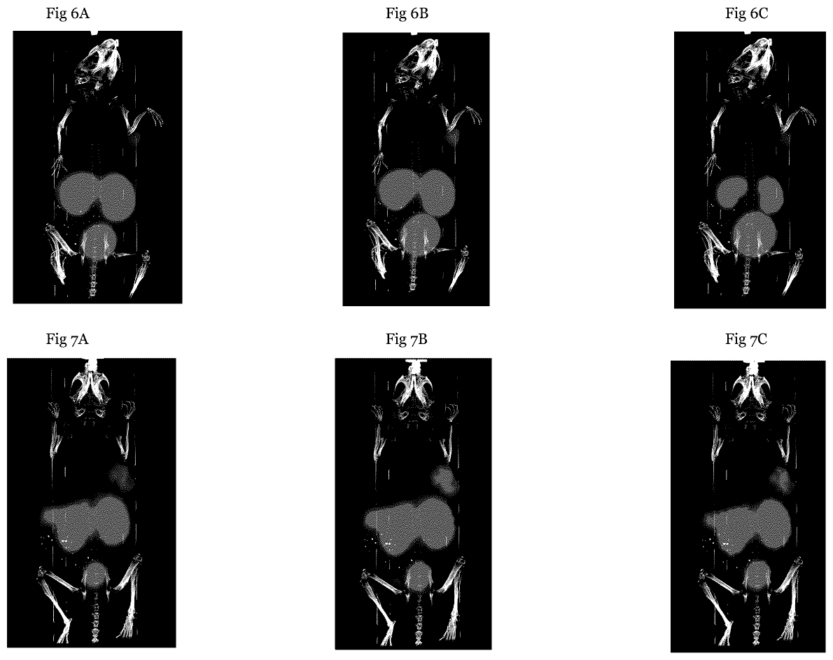

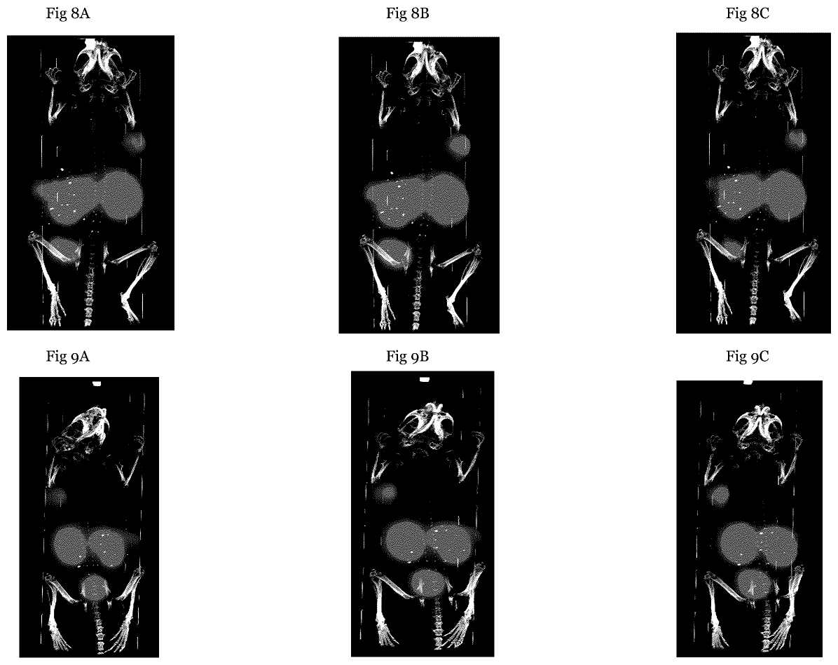

For μPET studies 60 pmol (3-10 MBq) [68Ga]-compounds were injected via tail vein into anesthetized mice. They were placed into the PET scanner and a dynamic scan over 130 min was performed. The results are shown in FIGS. 2-9. Tumor uptake was observed for all PSMA-binding ligands of the present invention containing heteroaromatic linker building blocks.

1. A compound of Formula I 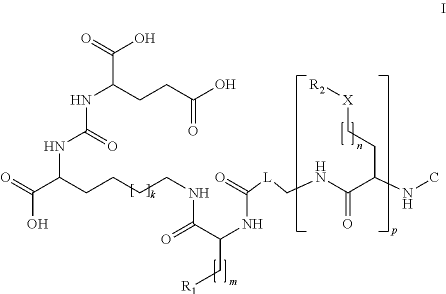
wherein
C is represented by any of the structures selected from the group consisting of
1,4,7,10-tetraazacyclododecane-1,4,7,10-tetraacetic acid (DOTA),
N,N″-bis[2-hydroxy-5-(carboxyethyl)benzyl]ethylenediamine-N,N″-diacetic acid (HBED-CC),
1,4-bis(carboxymethyl)-6-[bis(carboxymethyl)]amino-6-methylperhydro-1,4-diazepine (AAZTA),
1,4,7-triazacyclononane-1,4,7-triacetic acid (NOTA),
2-(4,7,10-tris(carboxymethyl)-1,4,7,10-tetraazacyclododecan-1-yl)pentanedioic acid (DOTAGA),
N⁴-[5-[[4-[[5-(acetylhydroxyamino)pentyl]amino]-1,4-dioxobutyl]hydroxyamino]pentyl]-N¹-(5-aminopentyl)-N¹-hydroxybutanediamide (DFO),
N¹-[5-(acetylhydroxyamino)pentyl]-N²⁶ -(5-aminopentyl)-N²⁶,5,16-trihydroxy-4,12,15,23-tetraoxo-5,11,16,22-tetraazahexacosanediamide (DFO*), or
5-[(2-mercapto-2-methylpropyl)[2-[(2-mercapto-2-methylpropyl)amino]ethyl]amino]pentanoic acid (BAT),
L is C1-C5-heteroaryl, optionally substituted with C1-C6 alkyl,
X is NH or CH₂,
k is 0, 1 or 2,
m is an integer from 1 to 7,
n is an integer from 0 to 6,
p is 0 or 1,
R₁ is phenyl, phenyl-NH—, benzothiophenyl or naphthyl,
wherein phenyl and phenyl-NH— is optionally substituted with 1, 2 or 3 groups each independently selected from halo, OH and NO₂, and
R₂ is aryl, aryl-C(O)—, or heteroaryl-C(O)—, optionally substituted with 1, 2 or 3 groups each independently selected from halo, OH, C1-C6-alkyl, OCH₃ and NO₂,
wherein C1-C5-heteroaryl contains 1, 2, 3 or 4 heteroatoms, each independently selected from N, O or S,
or a pharmaceutically acceptable salt thereof or a solvate thereof or a solvate of a salt thereof.

2. The compound of Formula I according to claim 1 that is a compound of Formula II 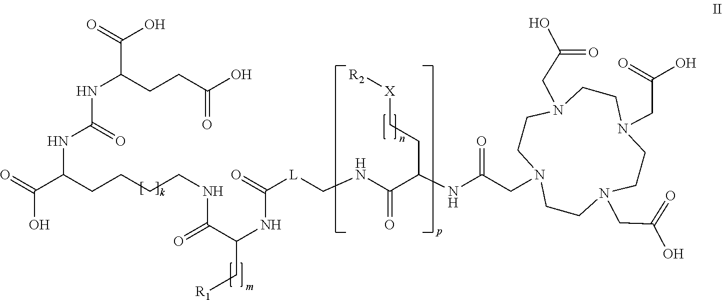
wherein
L, X, k, m, n, p, R₁ and R₂ are as defined in claim 1,
or a pharmaceutically acceptable salt thereof or a solvate thereof or a solvate of a salt thereof.

3. The compound of Formula I according to claim 1 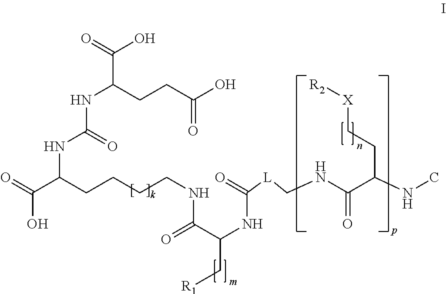
wherein
C, X, k, m, n, p, R₁ and R₂ are as defined in claim 1,
L is selected from the group consisting of isoxazolyl, pyridyl, thiazolyl and thiophenyl, optionally substituted with C1-C6 alkyl,
or a pharmaceutically acceptable salt thereof or a solvate thereof or a solvate of a salt thereof.

4. The compound of Formula I according to claim 1 that is a compound of Formula III 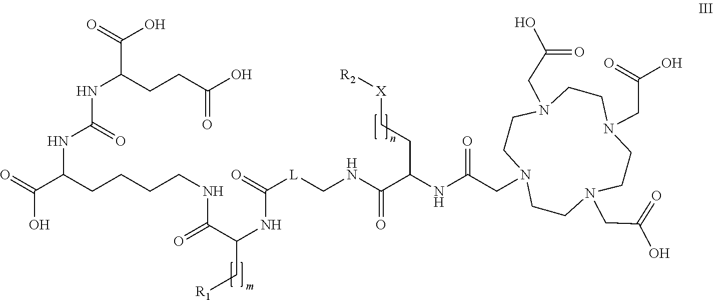
wherein
X, m, n, R₁ and R₂ are as defined in claim 1,
L is selected from the group consisting of isoxazolyl, pyridyl, thiazolyl and thiophenyl, optionally substituted with C1-C6 alkyl,
or a pharmaceutically acceptable salt thereof or a solvate thereof or a solvate of a salt thereof.

5. The compound of Formula I according to claim 1 that is a compound of Formula IV 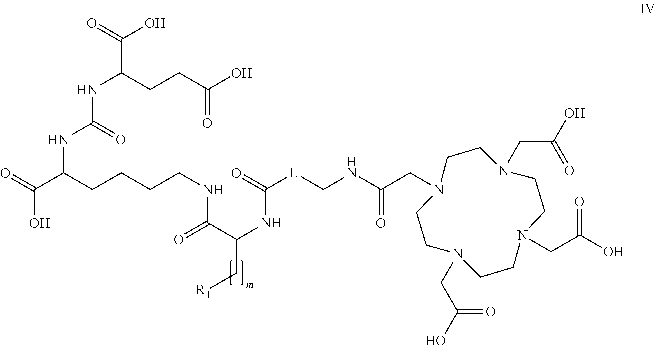
wherein
m and R₁ are as defined in claim 1,
L is selected from the group consisting of isoxazolyl, pyridyl, thiazolyl and thiophenyl, optionally substituted with C1-C6 alkyl,
or a pharmaceutically acceptable salt thereof or a solvate thereof or a solvate of a salt thereof.

6. The compound of Formula I according to claim 1 that is a compound of Formula V 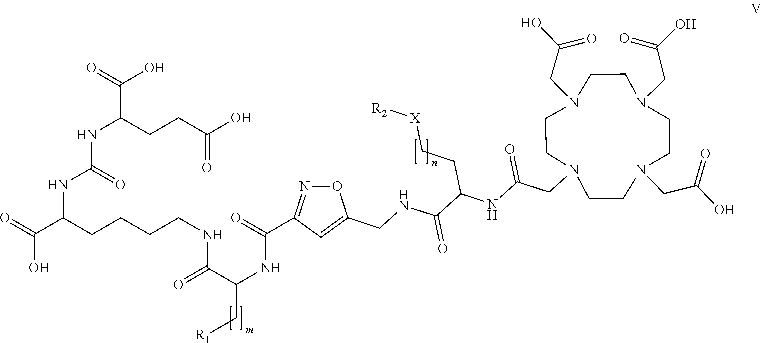
wherein
X, m, n, R₁ and R₂ are as defined in claim 1,
or a pharmaceutically acceptable salt thereof or a solvate thereof or a solvate of a salt thereof.

7. The compound of Formula I according to claim 1 that is a compound of Formula VI 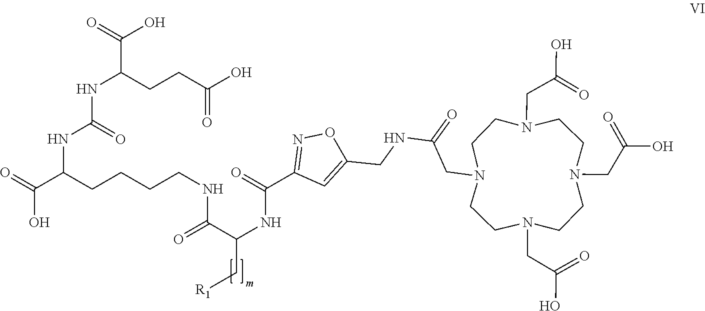
wherein
m and R₁ are as defined in claim 1,
or a pharmaceutically acceptable salt thereof or a solvate thereof or a solvate of a salt thereof.

8. The compound according to claim 1, wherein R₁ is benzothiophenyl or naphthyl and m is 1, or a pharmaceutically acceptable salt thereof or a solvate thereof or a solvate of a salt thereof.

9. The compound according to claim 1, that is a compound selected from the list of the following compounds: 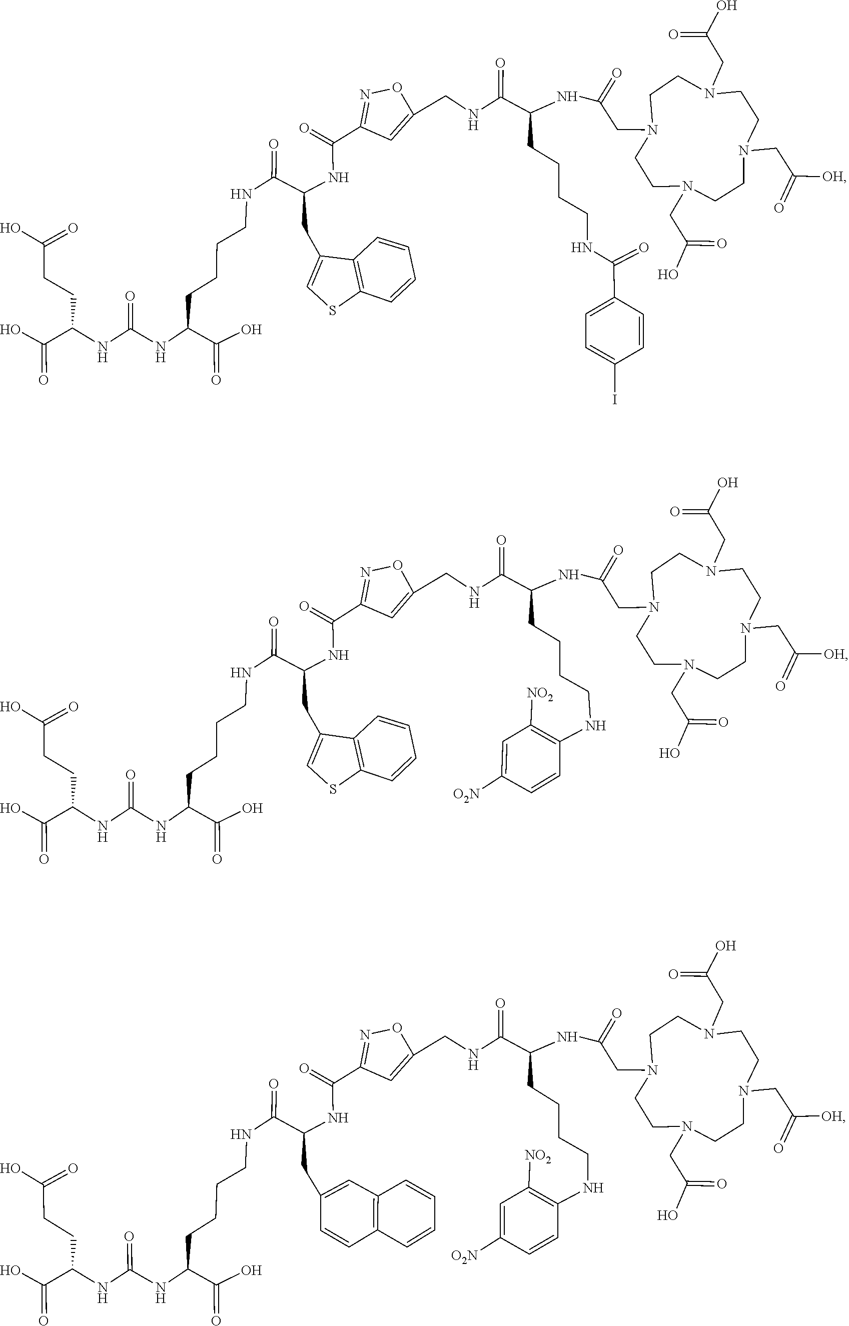 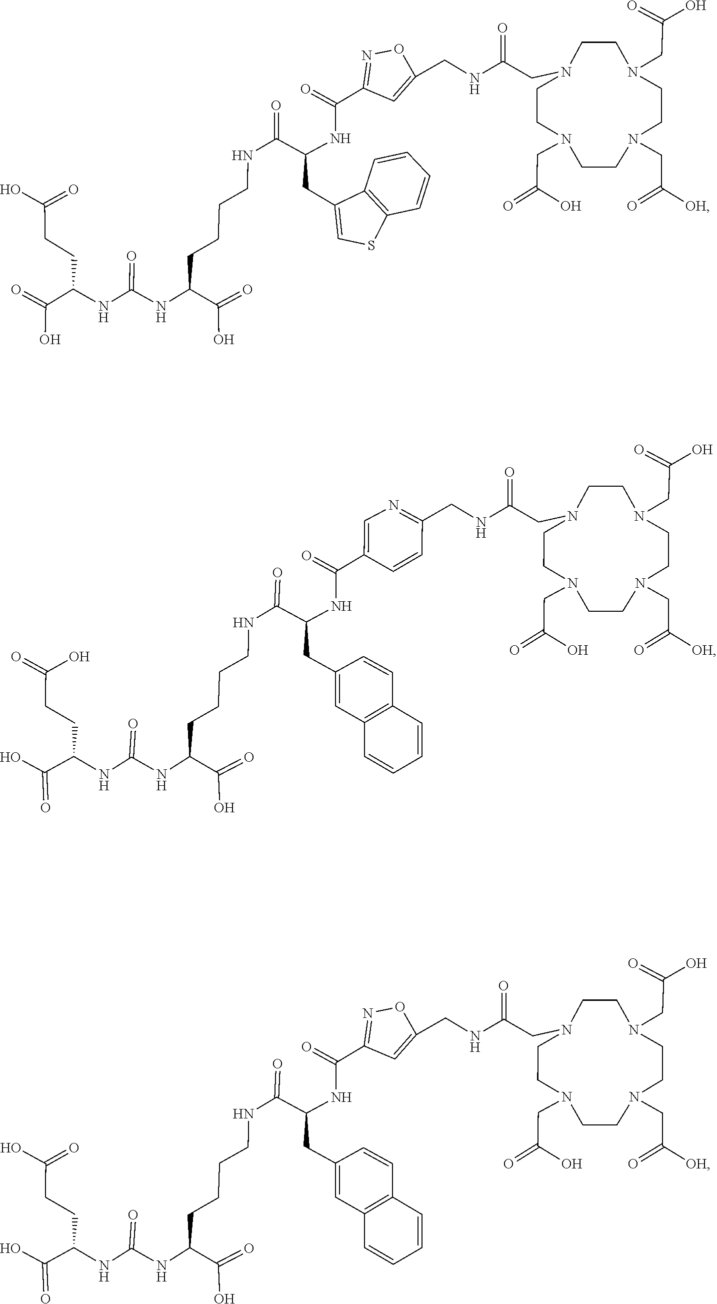 [SMILES: O=C(O)CC[C@H](NC(=O)N[C@@H](CCCCNC(=O)[C@H](Cc1csc2ccccc12)NC(=O)c1cc(CNC(=O)CN2CCN(CC(=O)O)CCN(CC(=O)O)CCN(CC(=O)O)CC2)on1)C(=O)O)C(=O)O ; O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)nc1)C(=O)O)C(=O)O ; O=C(O)CC[C@H](NC(=O)N[C@@H](CCCCNC(=O)[C@H](Cc1ccc2ccccc2c1)NC(=O)c1cc(CNC(=O)CN2CCN(CC(=O)O)CCN(CC(=O)O)CCN(CC(=O)O)CC2)on1)C(=O)O)C(=O)O] 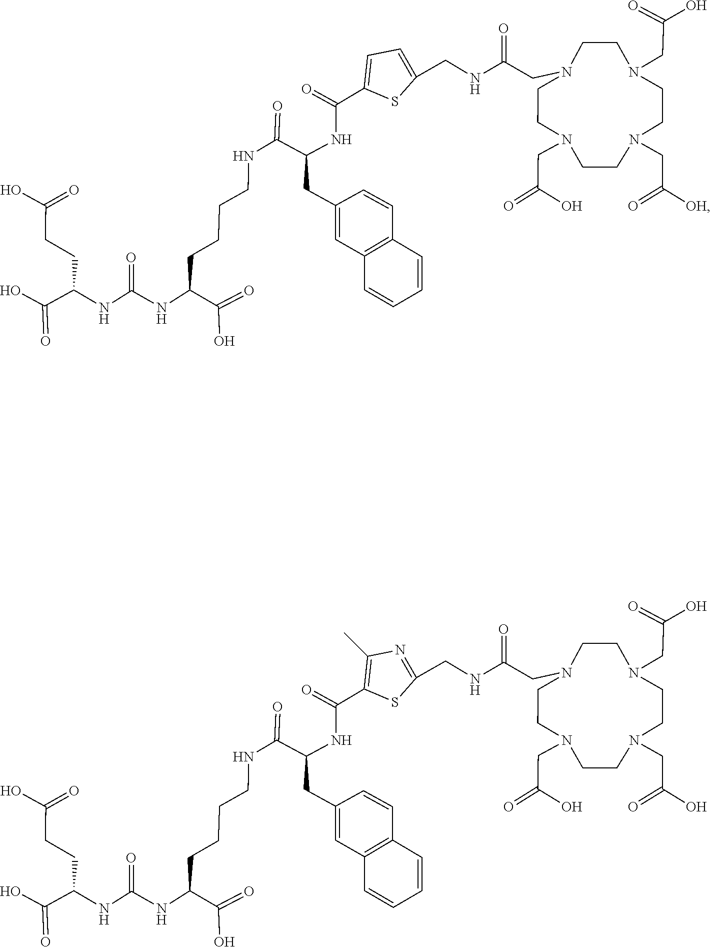 [SMILES: O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)s1)C(=O)O)C(=O)O ; Cc1nc(CNC(=O)CN2CCN(CC(=O)O)CCN(CC(=O)O)CCN(CC(=O)O)CC2)sc1C(=O)N[C@@H](Cc1ccc2ccccc2c1)C(=O)NCCCC[C@H](NC(=O)N[C@@H](CCC(=O)O)C(=O)O)C(=O)O]
or a pharmaceutically acceptable salt thereof or a solvate thereof or a solvate of a salt thereof.

10. A method for preparing a radiolabeled compound, comprising radiolabeling a compound according to claim 1 or a pharmaceutically acceptable salt thereof or a solvate thereof or a solvate of a salt thereof.

11. A metal complex comprising a radionuclide and a compound of claim 1 or a pharmaceutically acceptable salt thereof or a solvate thereof or a solvate of a salt thereof.

12. The metal complex according to claim 11, wherein the radionuclide is ¹¹¹In, ⁹⁰Y, ⁶⁸Ga, ¹⁷⁷Lu, ^{99m}Tc, ⁶⁴Cu, ⁶⁷Cu ¹⁵³Gd, ¹⁵⁵Gd, ¹⁵⁷Gd, ²¹³Bi, ²²⁵Ac, ⁸⁹Zr, ²⁰³Pb, ²¹²Pb.

13. The metal complex according to claim 11, wherein the radionuclide is ¹⁷⁷Lu.

14. The metal complex according to claim 11, wherein the radionuclide is ⁶⁸Ga.

15. The metal complex according to claim 11, wherein the radionuclide is ²²⁵Ac.

16. A pharmaceutical composition comprising a compound of claim 1 or a metal complex comprising a radionuclide and said compound, or a pharmaceutically acceptable salt, or a solvate thereof, or a solvate of a salt thereof, and a pharmaceutically acceptable carrier.

17. A metal complex according to claim 11 or a pharmaceutically acceptable salt thereof or a solvate thereof or a solvate of a salt thereof for use in a method of imaging in a patient.

18. A metal complex according to claim 11 or a pharmaceutically acceptable salt thereof or a solvate thereof or a solvate of a salt thereof for use in a method of diagnosing a cancer and/or metastasis thereof, optionally selected from the group of cancers that are positive for expression of PSMA, further optionally prostate cancer.

19. A metal complex according to claim 11 or a pharmaceutically acceptable salt thereof or a solvate thereof or a solvate of a salt thereof for use in a method for treating cancer and/or metastasis thereof, optionally selected from the group of cancers that are positive for expression of PSMA, further optionally prostate cancer.

20. A method of diagnosing cancer and/or metastasis thereof, optionally selected from the group of cancers that are positive for expression of PSMA, further optionally prostate cancer, comprising administering to an individual a metal complex according to claim 11 or a pharmaceutically acceptable salt thereof or a solvate thereof or a solvate of a salt thereof.

21. A method of treating cancer and/or metastasis thereof, optionally selected from the group of cancers that are positive for expression of PSMA, further optionally prostate cancer, comprising administering to an individual a metal complex according to claim 11 or a pharmaceutically acceptable salt thereof or a solvate thereof or a solvate of a salt thereof.
